(19) United States
(12) Patent Application Publication
(10) Pub. No.: US 2022/0275400 A1
(43) Pub. Date: Sep. 1, 2022

(54) METHODS FOR SCALABLE GENE INSERTIONS

(71) Applicant: THE TRUSTEES OF COLUMBIA UNIVERSITY IN THE CITY OF NEW YORK, New York, NY (US)

(72) Inventors: Alejandro Chavez, New York, NY (US); Brijesh Kumar Singh, New York, NY (US)

(21) Appl. No.: 17/637,710

(22) PCT Filed: Aug. 31, 2020
(86) PCT No.: PCT/US2020/048774

§ 371 Date: Feb. 23, 2022

Related U.S. Application Data
(60) Provisional application No. 62/894,067, filed on Aug. 30, 2019.

(51) Int. Cl. C12N 15/90; C12N 5/071; C12N 15/11; C12N 9/22; C12N 15/85

(52) U.S. Cl. CPC C12N 15/90; C12N 5/0687; C12N 15/11; C12N 9/22; C12N 15/85; C12N 2310/20; C12N 2800/80; C12N 2800/107

(57) ABSTRACT

The present invention relates to high throughput in vitro genetic manipulation. In particular, it relates to scalable CRISPR gene insertions in mammalian cells.

### RELATED APPLICATION DATA

This application claims priority to U.S. Provisional Application No. 62/894,067 filed on Aug. 30, 2019, which is hereby incorporated herein by reference in it its entirety for all purposes.

### FIELD OF THE INVENTION

The present invention relates to high throughput in vitro genetic manipulation. In particular, it relates to scalable CRISPR gene insertions in mammalian cells.

### BACKGROUND

Manipulating and selective labeling of endogenous proteins is essential to delineating the molecular mechanisms of cell and organismal biology. Techniques to enable these strategies are a fundamental cornerstone of biomedical research, however they are often inefficient, labor intensive, or imprecise. For example, protein depletion strategies using RNAi are susceptible to off-target effects in a concentration-dependent manner and gene knockout approaches require considerable investments in time and resources. Common antibody methods to label endogenous proteins are also problematic. It is estimated that a large fraction of available antibodies have limited utility due to unsuspected cross-reactivity to other proteins, lot-to-lot variability of quality, and improper validation of antibodies across the array of applications they are used for. Questionable antibodies are likely the key reagent driving disparate and irreproducible findings across laboratories, leading to a call for a solution to the large number of suspect antibodies currently flooding the market. Additionally, overexpression of recombinant protein to visualize protein localization and dynamics or to create dominant negative phenotypes is highly sensitive to the concentration of the expressed protein, available cellular docking sites, and unforeseen artifactual cellular effects.

Clustered regularly interspaced short palindromic repeats (CRISPR)-associated endonuclease Cas9-based strategies have great promise to enable highly precise genome editing to address many of the above limitations. CRISPR-Cas9 introduces double strand breaks (DSBs) at guide RNA (gRNA) specified genomic sites. The requirement for gRNA directed targeting of Cas9-mediated double stranded breaks is based on a “protospacer adjacent motif (PAM) sequence at the end of a 20-21 bp nucleotide gene specific sequence. The PAM sequence for Cas9 derived from Streptococcus pyogenes (the most common variant in use) is 5′-NGG-3′, where N represents any nucleotide. It is estimated this sequence is found on average about every 10 nucleotides (+ and − strand combined), opening up the potential to target essentially every gene. Double strand genomic breaks are repaired via two pathways in cells. Non-homology end joining (NHEJ) is the preferred pathway, which can introduce insertions or deletions (Indels) that introduce non-sense mutations during the repair process. Alternatively, DSBs can be repaired by the less frequent pathway of homology-directed repair (HDR). CRISPR methods are disclosed, for example, in U.S. Pat. No. 9,023,649 and U.S. Pat. No. 8,697,359, the disclosures of which are expressly incorporated here.

Both pathways (NHEJ and HDR) are currently utilized to manipulate endogenous proteins in cells or tissue via donor vectors to insert foreign sequences (payloads) into genes of interest (GOIs). Single cell labeling of endogenous proteins (SLENDR and viral (v)SLENDR) is based on HDR, using oligo or Adeno- Associated Virus (AAV) donors containing homologous gene-specific sequences of hundreds of basepairs flanking the DSB cut site to deliver sequences into a GOI (Mikuni et al., Cell 165:1803-1817 (2016); Nishiyama et al., Neuron 96:755-768 e755 (2017)). Alternatively, CRISPaint pairs NHEJ with a generalized donor vector that is linearized and integrated into the gene of interest (Schmid-Burgk et al., Nat Commun 7: 12338 (2016)).

Generic methods of doing targeted knock-ins at genome wide scales in lower eukaryotes (e.g., yeast) has been reported in Guo et al., Nat. Biotechnol. 2018, 36(6):540-546, which is incorporated herein by reference in its entirety. As the homologous recombination (HR) rates are lower in mammals, such as humans than in yeast, this method is not expected to work in mammalian cells, such as human cells.

Modular base-specific gene tagging has been reported in Schmid-Burgk et al., Nat. Commun. 2016, 7:12338, which is incorporated herein by reference in its entirety. However, this so-called CRISPaint method can only tag one gene at a time, as it requires a unique transfection for each targeted gene. These issues limit its potential applicability, especially for large scale applications.

Protein engineering allows enhanced production of chemicals, fuels, and medicine. To achieve high levels of protein production, protein synthesis mechanisms are typically transplanted into various host organisms from other organisms. Biosynthetic pathway construction and optimization requires large-scale manipulation of native and heterologous genes For several decades, the standard method of introducing or removing genes from organisms relied on gene targeting by PCR-generated marker cassettes. These techniques were often limited by the limited number of unique selection markers that work in an organism of interest. Limited selection markers limits the number of genes that can be removed and heterologous genes that can be inserted easily. Additionally, such techniques require successive rounds of screening to achieve desired organisms with multiple genes introduced or modified. Such techniques are time-consuming and highly inefficient. In addition to being not scalable, none of the above technologies have been demonstrated in mammalian cells.

### SUMMARY OF THE INVENTION

There has not been any scalable method of gene tagging in mammalian cells. While it can take months for the current technologies to tag many proteins of interest, the present method allows a large number (e.g., hundreds) of genes to be tagged within a similar time frame.

The present methods are scalable (e.g., double and triple gene tagging, etc.). For example, the method may be used to tag genes at library scales. The present methods may be used for protein engineering. The present methods may be used, e.g., to add an N or C-terminal protein tag (e.g., make a genome-wide library of cells that are YFP-tagged, degron-tagged, under inducible transcriptional control, FLAG-tagged, etc.), or to enable a promoter swap.

The present methods can be used for rapidly creating libraries of isogenic cell lines each containing a unique targeted knock-in. The method employs the RNA-guided endonuclease Cas9 plus a donor plasmid containing the DNA sequence (e.g., EYFP) to be fused to the target locus/gene, a gRNA against the target locus (i.e., target selector gRNA), and a gRNA against the donor plasmid (i.e., donor selector gRNA) (FIG. 1A-1B). Once all components are delivered into a cell, the genome-targeting gRNA directs Cas9 to cut within the gene we wish to fuse our DNA sequence of interest to, while the donor selector gRNA is used to linearize the circular donor construct (FIG. 2). During the repair of the DNA break at the target gene, the linearized donor can become inserted into the locus, which creates a fusion between the cut gene and the donor molecule. We are able to enrich for cells that have the donor molecule properly knocked into the genome by selecting for drug resistance, as our knocked-in sequence not only encodes a tag we wish to fuse to our gene of interest (e.g., YFP) but also a drug resistance gene. As Cas9 can cut DNA three base pairs upstream of the PAM sequence, we are able to use this knowledge to predict where within the donor plasmid we should cut such that when it fuses with the endogenous locus the inserted donor product will be in frame with the upstream target gene (FIG. 3).

To tag multiple genes within a population of cells, we first generate a pool of cells that each expresses a unique gRNA against one of the numerous genes we want to tag (FIG. 4). The population of cells is then transfected with Cas9, donor plasmid and donor selector gRNA (FIGS. 5A-5B). Following transfection, cells are selected with puromycin to enrich for those in which the donor molecule has been knocked into the proper locus. To obtain a homogenous clone for each knock-in, we sort individual cells into a single well of a multi-well plate and allow the single cells to proliferate and establish a clonal population (FIGS. 6A-6B). To determine the identity of each gene tagged within the various clonal populations, the gRNA within each well can be sequenced followed by PCR verification that the predicted junction between the gene of interest and the donor exists. If a more high-throughput approach for determining the identity of each clone is desired, a Cartesian Pooling-Coordinate Sequencing approach can be used. Besides tagging fluorescent proteins to our genes of interest, the present method can be used to fuse any DNA sequence of interest such as an epitope tag, small molecule regulated degron, or effector protein to our gene of interest (FIG. 7). Once we have our clonal lines, they can be used for any downstream assay of interest, such as observing changes in protein localization with environmental perturbation, discovering novel interacting partners or mapping DNA/RNA interactions in high-throughput (FIGS. 8A, 8B, 8C).

In one embodiment, to avoid any competition between the target selector gRNA and donor selector gRNA for Cas9 within the cell (FIG. 9A), two orthogonal Cas9 proteins may be used, with each Cas9 protein designed to interact with either the target selector or donor selector gRNA, thus eliminating competition between the gRNAs and Cas9. This strategy led to an improved efficiency of drug resistant cells showing the proper gene being tagged (FIG. 9B).

Besides knocking in a DNA sequence within a single defined locus within each cell in the cell population, the present methods can also be used to simultaneously generate a pool of cells in which each cell has two or more sequences knocked in at several defined genomic loci. To do this, a target selector construct that expresses a set of orthogonal gRNAs against two or more target sites of interest is first introduced into the cell (FIG. 10). We then perform serial rounds of transfection and drug selection to obtain our pool of multiple knock-in cells (FIGS. 11A-11B). Cells in which multiple genomic loci are tagged can then be used to study the dynamics between several proteins within live cells.

Disclosed herein are systems and methods for scalable gene insertions.

Accordingly, in a first aspect, the present invention provides a system for modifying a plurality of target sites in a mammalian cell, wherein the system comprises at least one type of vector comprising: (i) a plurality of donor DNAs; (ii) a plurality of first sequences encoding a plurality of first guide RNAs that hybridize to a plurality of target sites in the cell; (iii) a plurality of second sequences encoding a plurality of second guide RNAs that hybridize to the plurality of donor DNAs; and, (iv) a third sequence encoding a sequence-specific nuclease.

In various embodiments of the first aspect of the invention delineated herein, two, three, or all four of (i)-(iv) are present in the same vector. In various embodiments of the first aspect of the invention delineated herein, each of (i)-(iv) are present in different vectors.

In various embodiments of the first aspect of the invention delineated herein, the plurality of first sequences encoding a plurality of first guide RNAs hybridize to a plurality of different target sites in the cell.

In various embodiments of the first aspect of the invention delineated herein, the at least one type of vector comprises lentiviral vectors or plasmid vectors.

In various embodiments of the first aspect of the invention delineated herein, the at least one type of vector comprises at least one additional sequence encoding at least one additional sequence-specific nuclease. In various embodiments of the first aspect of the invention delineated herein, the sequence-specific nuclease and the at least one additional sequence-specific nuclease are different.

In various embodiments of the first aspect of the invention delineated herein, the at least one type of vector encodes two sequence-specific nucleases binding to the first guide RNAs or the second guide RNAs, respectively. In various embodiments of the first aspect of the invention delineated herein, at least one or all of the sequence-specific nucleases is a Cas nuclease. In various embodiments of the first aspect of the invention delineated herein, each of the sequence-specific nucleases is a Cas9 ortholog individually selected from the group consisting of Streptococcus pyogenes, Staphylococcus aureus, Steptococcus thermophilus, Lactobacillus gasseri, Francisella novicida, Wolinella succinogenes, Sutterella wadsworthensis, gamma proteobacterium, Neisseria meningitidis, Camplyobacteri jejuni, Fibrobacter succinogenes, Rhodobacter speaeroides, Thermus thermophilus, Pyrococcus pyogenes, and Rhodospirillum rubrum, or recombinant hybrids thereof, preferably wherein each Cas nuclease is a distinct ortholog from each other. In various embodiments of the first aspect of the invention delineated herein, at least one of the Cas nuclease is Streptococcus pyogenes Cas9 (SpCas9). In various embodiments of the first aspect of the invention delineated herein, the Cas nuclease and the second Cas nuclease are Streptococcus pyogenes Cas9 (SpCas9) and Staphylococcus aureus Cas9 (SaCas9), respectively. In various embodiments of the first aspect of the invention delineated herein, the Cas nuclease, the second Cas nuclease, and at least one additional Cas nuclease are Streptococcus pyogenes Cas9 (SpCas9), Staphylococcus aureus Cas9 (SaCas9), and Steptococcus thermophiles (StCas9), respectively.

In various embodiments of the first aspect of the invention delineated herein, the at least one type of vector further comprises a plurality of additional sequences encoding a plurality of selectable markers, optionally, comprising multiple distinct markers.

In various embodiments of the first aspect of the invention delineated herein, the plurality of selectable markers encodes a plurality of drug resistant markers, optionally multiple distinct drug resistant markers. In various embodiments of the first aspect of the invention delineated herein, the plurality of sequences encodes a plurality of drug resistant markers individually selected from the group consisting of puromycin resistant genes, blasticidin resistant genes, and or nourothricin resistant genes. In various embodiments of the first aspect of the invention delineated herein, the system further comprises a means for selecting for the selectable marker and a means for expanding after selection.

In various embodiments of the first aspect of the invention delineated herein, the plurality of donor DNAs comprises a plurality of sequences encoding for a plurality of gene insertions, optionally comprising multiple distinct insertions. In various embodiments of the first aspect of the invention delineated herein, the plurality of sequences encoding for a plurality of gene insertions encode proteins individually selected from the group consisting of an antibody tag, antibody-epitope tag, fluorescent protein tag, an affinity purification tag, a protein-protein interaction domain, a chemically induced protein-protein interaction domain, an enzyme, a recombinase site, a protein stability regulating tag, a spatial localization sequence, DNA/RNA targeting protein, or a combination thereof.

In various embodiments of the first aspect of the invention delineated herein, the system is for modifying a plurality of target sites in a population of mammalian cells, optionally, different mammalian cells.

In a second aspect, the present invention provides a system for modifying a target site in a population of mammalian cells, wherein the system comprises at least one type of vector comprising: (i) a donor DNA; (ii) a first sequence encoding a first guide RNA that hybridizes to a target site in the cell; (iii) a second sequence encoding a second guide RNA that hybridizes to the donor DNA; and, (iv) a third sequence encoding a sequence-specific nuclease.

In various embodiments of the second aspect of the invention delineated herein, two, three, or all four of (i)-(iv) are present in the same vector. In various embodiments of the second aspect of the invention delineated herein, each of (i)-(iv) are present in different vectors.

In various embodiments of the second aspect of the invention delineated herein, the at least one type of vector comprises lentiviral vectors or plasmid vectors.

In various embodiments of the second aspect of the invention delineated herein, the at least one type of vector further comprises at least one additional sequence encoding at least one additional sequence-specific nuclease. In various embodiments of the second aspect of the invention delineated herein, the sequence-specific nuclease and the at least one additional sequence-specific nuclease are different. In various embodiments of the second aspect of the invention delineated herein, the at least one type of vector encodes two sequence-specific nucleases binding to the first guide RNAs or the second guide RNAs, respectively.

In various embodiments of the second aspect of the invention delineated herein, at least one or all of the sequence-specific nucleases is a Cas nuclease. In various embodiments of the second aspect of the invention delineated herein, each of the sequence-specific nucleases is a Cas9 ortholog individually selected from the group consisting of Streptococcus pyogenes, Staphylococcus aureus, Steptococcus thermophilus, Lactobacillus gasseri, Francisella novicida, Wolinella succinogenes, Sutterella wadsworthensis, gamma proteobacterium, Neisseria meningitidis, Camplyobacteri jejuni, Fibrobacter succinogenes, Rhodobacter speaeroides, Thermus thermophilus, Pyrococcus pyogenes, and Rhodospirillum rubrum, or recombinant hybrids thereof, preferably wherein each Cas nuclease is a distinct ortholog from each other. In various embodiments of the second aspect of the invention delineated herein, the Cas nuclease is Streptococcus pyogenes Cas9 (SpCas9). In various embodiments of the second aspect of the invention delineated herein, the Cas nuclease and the second Cas nuclease are Streptococcus pyogenes Cas9 (SpCas9) and Staphylococcus aureus Cas9 (SaCas9), respectively.

In various embodiments of the second aspect of the invention delineated herein, the at least one type of vector further comprises at least one additional sequence encoding a selectable marker.

In various embodiments of the second aspect of the invention delineated herein, the sequence encoding for a selectable marker encodes a drug resistant marker. In various embodiments of the second aspect of the invention delineated herein, the sequence encoding for the drug resistant marker is selected from the group consisting of a puromycin resistant gene, blasticidin resistant gene, or nourothricin resistant gene. In various embodiments of the second aspect of the invention delineated herein, the system further comprises a means for selecting for the selectable marker and a means for expanding after selection.

In various embodiments of the second aspect of the invention delineated herein, the donor DNA comprises a sequence encoding for a gene insertion. In various embodiments of the second aspect of the invention delineated herein, the sequence encoding for a gene insertion encodes a protein selected from the group consisting of an antibody tag, antibody-epitope tag, fluorescent protein tag, an affinity purification tag, a protein-protein interaction domain, a chemically induced protein-protein interaction domain, an enzyme, a recombinase site, a protein stability regulating tag, a spatial localization sequence, or DNA/RNA targeting protein.

In a third aspect, the present invention provides a method for modifying a target site in a cell, the method comprising: contacting the cell with at least one type of vector comprising: (i) a donor DNA; (ii) a first sequence encoding a first guide RNA that hybridizes to a target site in the cell; (iii) a second sequence encoding a second guide RNA that hybridizes to the donor DNA; and, (iv) a third sequence encoding a sequence-specific nuclease.

In various embodiments of the third aspect of the invention delineated herein, the donor DNA is incorporated into the target site. In various embodiments of the third aspect of the invention delineated herein, upon incorporation into the target site, the cell expresses the donor DNA.

In various embodiments of the third aspect of the invention delineated herein, two, three, or all four of (i)-(iv) are present in the same vector. In various embodiments of the third aspect of the invention delineated herein, each of (i)-(iv) are present in different vectors.

In various embodiments of the third aspect of the invention delineated herein, the at least one type of vector comprises a lentiviral vector or a plasmid vector. In various embodiments of the third aspect of the invention delineated herein, the vector comprises a plasmid vector. In various embodiments of the third aspect of the invention delineated herein, the vector comprises a lentiviral vector.

In various embodiments of the third aspect of the invention delineated herein, the at least one type of vector comprises at least one additional sequence encoding at least one additional sequence-specific nuclease. In various embodiments of the third aspect of the invention delineated herein, the sequence-specific nuclease and the at least one additional sequence-specific nuclease are different. In various embodiments of the third aspect of the invention delineated herein, the at least one type of vector encodes two sequence-specific nucleases binding to either the first guide RNA or the second guide RNA, respectively.

In various embodiments of the third aspect of the invention delineated herein, at least one or all of the sequence-specific nuclease is a Cas nuclease. In various embodiments of the third aspect of the invention delineated herein, each of the sequence-specific nuclease is a Cas9 ortholog individually selected from the group consisting of Streptococcus pyogenes, Staphylococcus aureus, Steptococcus thermophilus, Lactobacillus gasseri, Francisella novicida, Wolinella succinogenes, Sutterella wadsworthensis, gamma proteobacterium, Neisseria meningitidis, Camplyobacteri jejuni, Fibrobacter succinogenes, Rhodobacter speaeroides, Thermus thermophilus, Pyrococcus pyogenes, and Rhodospirillum rubrum, or recombinant hybrids thereof, preferably wherein if more than one Cas nuclease each Cas nuclease is a distinct ortholog from each other. In various embodiments of the third aspect of the invention delineated herein, the Cas nuclease is Streptococcus pyogenes Cas9 (SpCas9). In various embodiments of the third aspect of the invention delineated herein, the Cas nuclease is Streptococcus pyogenes Cas9 (SpCas9) and an additional Cas nuclease is Staphylococcus aureus Cas9 (SaCas9).

In various embodiments of the third aspect of the invention delineated herein, the method is for modifying a target site in a population of mammalian cells.

In various embodiments of the third aspect of the invention delineated herein, the at least one type of vector further comprises at least one additional sequence encoding for a selectable marker. In various embodiments of the third aspect of the invention delineated herein, the at least one type of vector further comprises at least two additional sequences encoding for selectable markers. In various embodiments of the third aspect of the invention delineated herein, the at least one additional selectable marker and at least one second additional selectable marker are different. In various embodiments of the third aspect of the invention delineated herein, the method further comprises selecting the cells for the selectable marker and expanding the cells. In various embodiments of the third aspect of the invention delineated herein, the sequence encoding a selectable marker encodes a drug resistant marker gene. In various embodiments of the third aspect of the invention delineated herein, the sequence encoding a drug resistant marker is selected from the group consisting of a puromycin resistant gene, blasticidin resistant gene, or nourothricin resistant gene. In various embodiments of the third aspect of the invention delineated herein, selecting for the selectable marker comprises treating the cells with a corresponding selective agent. In various embodiments of the third aspect of the invention delineated herein, the selective agent is a drug. In various embodiments of the third aspect of the invention delineated herein, the drug is puromycin, blasticidin, or nourothricin.

In various embodiments of the third aspect of the invention delineated herein, the method further comprises at least one additional round of selecting for a selectable marker.

In various embodiments of the third aspect of the invention delineated herein, the donor DNA comprises a sequence encoding for a gene insertion. In various embodiments of the third aspect of the invention delineated herein, the sequence encoding for a gene insertion encodes a protein selected from the group consisting of an antibody tag, antibody-epitope tag, fluorescent protein tag, affinity purification tag, a protein-protein interaction domain, a chemically induced protein-protein interaction domain, an enzyme, a recombinase site, a protein stability regulating tag, a spatial localization sequence, or a DNA/RNA targeting protein.

In various embodiments of the third aspect of the invention delineated herein, (ii) optionally, contacts the cell prior to (i), (iii), (iv).

In a fourth aspect, the present invention provides a method for modifying a plurality of target sites in a mammalian cell, the method comprising: contacting the cell with at least one type of vector comprising: (i) a plurality of donor DNAs; (ii) a plurality of first sequences encoding a plurality of first guide RNAs that hybridize to a plurality of target sites in the cell; (iii) a plurality of second sequences encoding a plurality of second guide RNAs that hybridize to the plurality of donor DNAs; and,(iv) a third sequence encoding as sequence-specific nuclease.

In various embodiments of the fourth aspect of the invention delineated herein, the plurality of donor DNAs are incorporated in the plurality of target sites. In various embodiments of the fourth aspect of the invention delineated herein, upon incorporation into the target sites, the cell expresses the plurality of donor DNA.

In various embodiments of the fourth aspect of the invention delineated herein, two, three, or all four of (i)-(iv) are present in the same vector. In various embodiments of the fourth aspect of the invention delineated herein, each of (i)-(iv) are present in different vectors.

In various embodiments of the fourth aspect of the invention delineated herein, the plurality of first sequences encoding a plurality of first guide RNAs hybridize to a plurality of different target sites in the cell.

In various embodiments of the fourth aspect of the invention delineated herein, the at least one type of vector comprises a lentiviral vector or a plasmid vector. In various embodiments of the fourth aspect of the invention delineated herein, the vector comprises a plasmid vector. In various embodiments of the fourth aspect of the invention delineated herein, the vector comprises a lentiviral vector.

In various embodiments of the fourth aspect of the invention delineated herein, the at least one type of vector comprises at least one additional sequence encoding at least one additional sequence-specific nuclease. In various embodiments of the fourth aspect of the invention delineated herein, the sequence-specific nuclease and the at least one additional sequence-specific nuclease are different. In various embodiments of the fourth aspect of the invention delineated herein, the at least one type of vector encodes two sequence-specific nucleases binding to first guide RNAs and second guide RNAs, respectively. In various embodiments of the fourth aspect of the invention delineated herein, at least one or all of the sequence-specific nucleases is a Cas nuclease. In various embodiments of the fourth aspect of the invention delineated herein, each of the sequence-specific nucleases is a Cas9 ortholog individually selected from the group consisting of Streptococcus pyogenes, Staphylococcus aureus, Steptococcus thermophilus, Lactobacillus gasseri, Francisella novicida, Wolinella succinogenes, Sutterella wadsworthensis, gamma proteobacterium, Neisseria meningitidis, Camplyobacteri jejuni, Fibrobacter succinogenes, Rhodobacter speaeroides, Thermus thermophilus, Pyrococcus pyogenes, and Rhodospirillum rubrum, or recombinant hybrids thereof, preferably wherein if more than one Cas nuclease each Cas nuclease is a distinct ortholog from each other. In various embodiments of the fourth aspect of the invention delineated herein, the Cas nuclease is Streptococcus pyogenes Cas9 (SpCas9). In various embodiments of the fourth aspect of the invention delineated herein, the Cas nuclease is Streptococcus pyogenes Cas9 (SpCas9) and the additional Cas nuclease is Staphylococcus aureus Cas9 (SaCas9). In various embodiments of the fourth aspect of the invention delineated herein, at least one additional Cas nuclease is Streptococcus thermophile (StCas9).

In various embodiments of the fourth aspect of the invention delineated herein, the method is for modifying a plurality of target sites in a population of mammalian cells.

In various embodiments of the fourth aspect of the invention delineated herein, the at least one type of vector further comprises a plurality of additional sequences encoding for a plurality of selectable markers, optionally comprising multiple distinct markers. In various embodiments of the fourth aspect of the invention delineated herein, the plurality of additional sequences encoding for a plurality of selectable markers, encodes a plurality of drug resistant markers, optionally multiple distinct drug resistant markers. In various embodiments of the fourth aspect of the invention delineated herein, the method further comprises selecting for the selectable marker and expanding the cells. In various embodiments of the fourth aspect of the invention delineated herein, the plurality of sequences encoding a plurality of drug resistant markers are individually selected from the group consisting of a puromycin resistant gene, blasticidin resistant gene, or nourothricin resistant gene. In various embodiments of the fourth aspect of the invention delineated herein, selecting for the selectable marker comprises treating the cells with a corresponding selective agent. In various embodiments of the fourth aspect of the invention delineated herein, selective agent is a drug. In various embodiments of the fourth aspect of the invention delineated herein, the drug is puromycin, blasticidin, or nourothricin. In various embodiments of the fourth aspect of the invention delineated herein, the method further comprises at least one additional round of selecting for a selectable marker.

In various embodiments of the fourth aspect of the invention delineated herein, the plurality of donor DNAs comprises a plurality of sequences encoding for a plurality of gene insertions, optionally comprising multiple distinct insertions. In various embodiments of the fourth aspect of the invention delineated herein, the plurality of sequences encoding a plurality of gene insertions encodes a protein selected from the group consisting of an antibody tag, antibody-epitope tag, fluorescent protein tag, affinity purification tag, a protein-protein interaction domain, a chemically induced protein-protein interaction domain, an enzyme, a recombinase site, a protein stability regulating tag, a spatial localization sequence, DNA/RNA targeting protein.

In various embodiments of the fourth aspect of the invention delineated herein, (ii) optionally contacts the cell prior to (i), (iii), (iv).

In a fifth aspect, the present invention provides a plurality of mammalian cells, comprising one or more types of vectors encoding two or more first guide RNAs (gRNAs) that hybridize to one or more target sites in the cell.

In various embodiments of the fifth aspect of the invention delineated herein, the present invention further provides a system comprising a plurality of mammalian cells, comprising one or more types of vectors encoding two or more first guide RNAs (gRNAs) that hybridize to one or more target sites in the cell; and at least one type of vector comprising: (i) a plurality of donor DNAs; (ii) a plurality of second sequences encoding a plurality of second guide RNAs that hybridize to the plurality of donor DNAs; and, (iii) a fourth sequence encoding a sequence-specific nuclease.

In various embodiments of the fifth aspect of the invention delineated herein, two or all three of (i)-(iii) are present in the same vector. In various embodiments of the fifth aspect of the invention delineated herein,wherein each of (i)-(iii) are present in different vectors.

In various embodiments of the fifth aspect of the invention delineated herein, the two or more first sequences encoding two or more first guide RNAs hybridize to two or more different target sites in the cell.

In various embodiments of the fifth aspect of the invention delineated herein, the at least one type of vector comprises lentiviral vectors or plasmid vectors.

In various embodiments of the fifth aspect of the invention delineated herein, the at least one type of vector comprises at least one additional sequence encoding at least one additional sequence-specific nuclease. In various embodiments of the fifth aspect of the invention delineated herein, the sequence-specific nuclease and the at least one additional sequence-specific nuclease are different. In various embodiments of the fifth aspect of the invention delineated herein, the at least one type of vector encodes two sequence-specific nucleases binding to first guide RNAs or second guide RNAs, respectively.

In various embodiments of the fifth aspect of the invention delineated herein, at least one or all of the sequence-specific nucleases is a Cas nuclease. In various embodiments of the fifth aspect of the invention delineated herein, each of the sequence specific nuclease is a Cas9 ortholog individually selected from the group consisting of Streptococcus pyogenes, Staphylococcus aureus, Steptococcus thermophilus, Lactobacillus gasseri, Francisella novicida, Wolinella succinogenes, Sutterella wadsworthensis, gamma proteobacterium, Neisseria meningitidis, Camplyobacteri jejuni, Fibrobacter succinogenes, Rhodobacter speaeroides, Thermus thermophilus, Pyrococcus pyogenes, and Rhodospirillum rubrum, or recombinant hybrids thereof, preferably wherein each Cas nuclease is a distinct ortholog from each other. In various embodiments of the fifth aspect of the invention delineated herein, the Cas nuclease is Streptococcus pyogenes Cas9 (SpCas9). In various embodiments of the fifth aspect of the invention delineated herein, the additional Cas nuclease is Streptococcus pyogenes Cas9 (SpCas9). In various embodiments of the fifth aspect of the invention delineated herein, at least one additional Cas nuclease is Steptococcus thermophiles (StCas9).

In various embodiments of the fifth aspect of the invention delineated herein, the at least one type of vector further comprises a plurality of additional sequences encoding a plurality of selectable markers, optionally, comprising multiple distinct markers. In various embodiments of the fifth aspect of the invention delineated herein, the plurality of sequences encoding for a plurality of selectable markers comprises encoding for a plurality of drug resistant markers, optionally multiple distinct drug resistant markers. In various embodiments of the fifth aspect of the invention delineated herein, the plurality of sequences encoding for a plurality of drug resistant markers are individually selected from the group consisting of puromycin resistant genes, blasticidin resistant genes, and or nourothricin resistant genes. In various embodiments of the fifth aspect of the invention delineated herein, the system further comprises a means for selecting for the selectable marker and a means for expanding after selection.

In various embodiments of the fifth aspect of the invention delineated herein, the plurality of donor DNAs comprises a plurality of sequences encoding for a plurality of gene insertions, optionally comprising multiple distinct insertions. In various embodiments of the fifth aspect of the invention delineated herein, the plurality of sequences encoding for a plurality of gene insertions encode proteins individually selected from the group consisting of an antibody tag, antibody-epitope tag, fluorescent protein tag, an affinity purification tag, a protein-protein interaction domain, a chemically induced protein-protein interaction domain, an enzyme, a recombinase site, a protein stability regulating tag, a spatial localization sequence, DNA/RNA targeting protein.

Other features and advantages of the invention will be apparent from the following detailed description and drawings.

### BRIEF DESCRIPTION OF THE FIGURES

FIG. 1A-1B. depicts an overview of the components of the protein tagging systems and methods described herein. FIG. 1A) Briefly, a host cell is selected. 293T cells are used as a generic cellular chassis in these examples, however any suitable mammalian cells are envisioned for use as the host cells. Next, suitable plasmids encoding the desired target selector gRNA, a donor selector gRNA, the Cas9 protein and a donor plasmid are obtained. Then, the host cells are transfected with the set of plasmids. Once the constructs are within the host cells, selective tagging of the locus or loci, of interest is/are obtained. FIG. 1B depicts exemplary microscopic images of the protein tagging systems and methods described herein. From left to right, the panels depict successful fluorescent tagging of the Histone H4 protein via insertion at the HIST1H4C gene locus, the gamma-actin protein via insertion at the ACTG1 gene, and the tubulin beta-chain via insertion at the TUBB gene, respectively.

FIG. 2 depicts the process by which the sequence of interest is introduced into the host cell's genome. Once all constructs are within the cell, spCas9 complexes with the target selector gRNA and makes a double strand break within the host cell's target gene. Separately, spCas9 also complexes with the donor selector gRNA, makes a break within the donor's sequence of interest and linearizes the donor plasmid. Once linearized the donor plasmid is able to become incorporated within the host's target genomic cut site.

FIG. 3 depicts the basis of donor plasmid selection. To ensure that the content of the donor construct is in frame with the target gene, special attention must be paid to the reading frame of the target gene. There are three potential reading frames (Frame-0, Frame-1, Frame-2), a donor is selected such that when it is cut by Cas9 and knocked into the target site it will be in the same frame as the target gene. If an inappropriate donor plasmid is selected it will lead to the knock-in product being out of frame and thus the fusion protein will fail to properly express.

FIG. 4 depicts an overview of an embodiment of the systems and methods described herein wherein a library of isogenic cell lines with precise genomic insertions, a target selector cell library, is created. First, a population of target selector plasmids is generated in which each plasmid contains a gRNA against a single unique genomic site that is desired to perform the targeted knock-in to. Then, the library of target selectors is converted into a population of lentiviruses and that are then used to infect host cells with the virus at a low multiplicity of infection to ensure that each cell receives at most a single gRNA. Cells that were properly transduced are then selected via a drug marker that is in cis to the gRNA (blasticidin resistance), and expanded. At the end of this stage, the target selector cell library is generated.

FIG. 5A-FIG. 5B depicts an overview of an embodiment of the systems and methods described herein wherein the target selector cell library is utilized to perform targeted knock-in on multiple genomic loci at once. FIG. 5A depicts the addition of Cas9, donor plasmid and donor selector gRNA to the mixed pool of target selector cell library and undergo one large en masse electroporation or other method of transfection. Cells with proper targeted knock-in begin to express a drug resistance marker which can be used to enrich for cells that have been appropriately modified by treating the transfected cell pool with the drug and expanding the surviving cell pool to obtain the tagged cell pool. FIG. 5B depicts successful fluorescent tagging expressed by the tagged cell pool wherein the tagged cell pool emits fluorescent light at various wavelengths, where the goal was to knock-in YFP into various genes, with each cell knocking-in YFP into a single gene.

FIG. 6A-FIG. 6B depicts an overview of an embodiment of the systems and methods described herein wherein a pure isogenic population is obtained. FIG. 6A illustrates that the mixed pool of successfully knock-in cells, are harvested and single cell sorted, wherein a single cell is plated into each well of a microwell plate. These cells are then cultured until the wells become confluent. Once expanded, a homogenous isogenic population of cells is obtained with each well having a different gene tagged with YFP. FIG. 6B depicts representative images from several different wells each with a different gene tagged at the C-terminus with YFP using the methods and systems described herein.

FIG. 7 depicts some examples of genetic insertions that may be inserted using the methods and systems described herein. The described plastic methods and systems can be used to insert any DNA sequence into any defined genomic locus. Possible knock-in genes include epitope tags, small molecule regulated degrons, or effector proteins in-frame with the target gene.

FIGS. 8A, 8B, and 8C depicts visual representations of exemplary applications of the methods and systems described herein. FIG. 8A depicts one embodiment wherein the methods and systems described are utilized to generate multi-tagged cells used to monitor the effect of an environmental perturbation or drug on the localization or expression level of each of the tagged proteins. FIG. 8B depicts another embodiment wherein the methods and systems described herein are utilized to generate epitope tagged cells used to isolate and identify interacting proteins or protein complexes. FIG. 8C depicts another embodiment wherein the methods and systems described herein are utilized to isolate and identify DNA binding proteins and gene regulators.

FIGS. 9A and 9B depict enhancement of targeted knock-in using orthogonal Cas9 proteins. FIG. 9A depicts a representation When a single Cas9 protein is used, it may be limiting during the reaction and be unevenly partitioned between the target selector and donor selector gRNA, thus leading to only one of the two loci being cut and low efficiency of integration. By using two orthogonal Cas9 proteins each able to interact with only the target selector gRNA or only the donor selector gRNA competition is prevented between the gRNAs and thus more efficient cutting of the donor and the target locus of interest is observed. FIG. 9B illustrates improvement in gene targeting efficiency using saCas9 and spCas9 as compared to using just spCas9.

FIG. 10 depicts an overview of an embodiment of the present invention for tagging more than one genomic locus using the methods/systems described herein. In this approach the initial construct that is integrated contains gRNAs against more than one genomic site with each gRNA being specific to a particular Cas9 variant. The construct is then introduced into cells and stably transduced cells are selected using the drug marker that is in cis to the gRNAs within the delivered construct.

FIGS. 11A and B illustrates multiplex locus knock-in within a single cell and applications of the technology. FIG. 11A depicts an overview of an embodiment of the present invention for tagging more than one genomic locus using the methods/systems described herein. Once a set of cells each expressing a unique set of gRNAs is established, repeat rounds of transfection and drug selection are applied. With each round one additional locus is tagged. This examples demonstrates that a single pair of loci may be targeted, however, in use each cell at the end of the process would have different combinations of loci tagged. FIG. 11B depicts an exemplary embodiment of the applications of the present invention. By tagging multiple genomic loci one can study the in vivo dynamics between sets of proteins.

### DETAILED DESCRIPTION OF THE INVENTION

### Definitions

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein an “antibody” refers to IgG, IgM, IgA, IgD or IgE molecules or antigen-specific antibody fragments thereof (including, but not limited to, a Fab, F(ab′)2, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria. In another example, an antibody includes two heavy (H) chain variable regions and two light (L) chain variable regions. It should be noted that a VH region (e.g. a portion of an immunoglobulin polypeptide is not the same as a VH segment, which is described elsewhere herein). The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

As described herein, an “antigen” is a molecule that is bound by a binding site on an antibody. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

“Binding” as used herein (e.g. with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower Kd.

“Binding region” as used herein refers to the region within a nuclease target region that is recognized and bound by the nuclease.

The term “Cas nuclease” as used herein describes CRISPR-associated protein, which is an RNA-guided endonuclease that is directed towards a desired genomic target when complexed with an appropriately designed small guide RNA (“gRNA”). An example of a Cas nuclease is Cas9 which is CRISPR-associated protein 9. gRNAs comprise approximately a 20-nucleotide sequence (the protospacer), which is complementary to the genomic target sequence. Next to the genomic target sequence is a 3′ protospacer-associated motif (“PAM”), which is required for Cas9 binding. In the case of Streptococcus Pyogenes Cas9 (SpCas9), this has the sequence NGG. Other sequences are as described herein and as known in the art. Upon binding the DNA target, Cas9 cleaves both strands of DNA, thereby stimulating repair mechanisms that can be exploited to modify the locus of interest.

“Cleave” or “cleavage” as used herein means the act of breaking the covalent sugar- phosphate bond between two adjacent nucleotides within a polynucleotide. In the case of a double-stranded polynucleotide, a covalent sugar-phosphate bond on both strands will be broken, unless otherwise specified.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein means a nucleic acid can Watson-Crick (e.g., A-T/U and C-G) or Hoogsteen base pair between nucleotides or nucleotide analogs of nucleic acid molecules. “Complementarity” refers to a property shared between two nucleic acid sequences, such that when they are aligned antiparallel to each other, the nucleotide bases at each position will be complementary.

“Donor vector”, “donor template” and “donor DNA” as used interchangeably herein refers to a double-stranded DNA fragment or molecule that includes the insert being introduced into the genomic DNA. The donor vector may encode a fully-functional protein, a partially-functional protein or a short polypeptide. The donor vector may also encode an RNA molecule.

The terms “engineered”, “constructed” or “designed” as used interchangeable herein, refers to the aspect of having been manipulated by the hand of man. As is common practice and is understood by those in the art, progeny and copies of an engineered polynucleotide (and/or cells or animals comprising such polynucleotides) are typically still referred to as “engineered” even though the actual manipulation was performed on a prior entity.

“Functional” and “full-functional” as used herein describes protein that has biological activity. A “functional gene” refers to a gene transcribed to mRNA, which is translated to a functional protein.

“Genetic construct” as used herein refers to the DNA or RNA molecules that comprise a nucleotide sequence that encodes a protein or an RNA molecule. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Genome editing” as used herein refers to changing a gene. Genome editing may include correcting or restoring a mutant gene. Genome editing may include knocking out a gene, such as a mutant gene or a normal gene. Genome editing may be used to introduce a label onto a protein.

The term “gRNA” as used herein describes small guide RNAs and is used interchangeably with the term “sgRNA.” Cas9 associated guide RNA's for mammalian systems may be generated through methods known in the art, for example as described in Nageshwaran, S., et al. (2018). CRISPR Guide RNA Cloning for Mammalian Systems. Journal of Visualized Experiments, (140). doi:10.3791/57998, the entirety of which is incorporated by reference herein.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent Identity may be performed manually or by using a computer sequence algorithm such as BLAST or BLAST 2.0.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker, an “increase” is a statistically significant increase in such level.

“Mismatch” as used herein means a nucleotide cannot form a Watson-Crick (e.g., A-T/U and C-G) or Hoogsteen base pair with another nucleotide on the opposite strand of a double- stranded polynucleotide or with another nucleotide from a different polynucleotide.

The term “plurality” as used herein means a number greater than one.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that can introduce random micro-insertions and micro-deletions (indels) at the DNA breakpoint This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

The terms “nucleic acid” or “oligonucleotide” or “polynucleotide” as used interchangeably herein means at least two nucleotides upwards of any length, either ribonucleotides or deoxyribonucleotides, covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or hybrids, or a polymer, where the nucleic acid may contain combinations of deoxyribo- and ribo-nucleotides, and combinations of bases including uracil, adenine, thymine, cytosine, guanine, inosine, xanthine hypoxanthine, isocytosine, isoguanine, purine and pyrimidine bases or other natural, chemically or biochemically modified, non-natural, or derivatized nucleotide bases. Nucleic acids may be obtained by chemical synthesis methods or by recombinant methods. “Oligonucleotide” generally refers to polynucleotides of between about 3 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

As used herein in connection with binding moeities, the term “orthogonal” means that two or more binding moieties indicated to be orthogonal to each other do not bind at a significant level to the same complementary binding pair member, i.e., they recognize different binding sites on different molecules.

“Promoter” as used herein means a synthetic or naturally-derived nucleic acid sequence which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

“Reading frame”, “Open Reading Frame” or “Coding Frame” as used herein interchangeably means a grouping of three successive bases in a sequence of DNA that potentially constitutes the codons for specific amino acids during translation into a polypeptide.

As used herein the term “sequence-specific nuclease” refers to programmable nucleases that enable genome editing by cleaving DNA at specific genomic loci, signaling DNA damage and recruiting endogenous repair machinery for either NHEJ or HDR to the cleaved site to mediate genome editing. Sequence-specific nucleases can be endonucleases, exonuclease, or both. The term “endonuclease” refers to enzymes that cleave the phosphodiester bond within a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An endonuclease may cut a polynucleotide symmetrically, leaving “blunt” ends, or in positions that are not directly opposing, creating overhangs, which may be referred to as “sticky ends.” The methods and compositions described herein may be applied to cleavage sites generated by endonucleases. In some alternatives of the system, the system can further provide nucleic acids that encode an endonuclease, such as CRISPR-associated protein (Cas), an Argonaute protein (AGO), TAL Effector Nuclease” (TALEN), or a meganuclease such as MegaTAL, or a fusion protein comprising a domain of an endonuclease, for example, Cas9, Ago, TALEN, or MegaTAL, or one or more portion thereof. Ago is a These examples are not meant to be limiting and other endonucleases and alternatives of the system and methods comprising other endonucleases and variants and modifications of these exemplary alternatives are possible without undue experimentation. All such variations and modifications are within the scope of the current teachings. The term “exonuclease” refers to enzymes that cleave phosphodiester bonds at the end of a polynucleotide chain via a hydrolyzing reaction that breaks phosphodiester bonds at either the 3′ or 5′ end. The polynucleotide may be double-stranded DNA (dsDNA), single-stranded DNA (ssDNA), RNA, double-stranded hybrids of DNA and RNA, and synthetic DNA (for example, containing bases other than A, C, G, and T). The term “5′ exonuclease” refers to exonucleases that cleave the phosphodiester bond at the 5′ end. The term “3′ exonuclease” refers to exonucleases that cleave the phosphodiester bond at the 3′ end. Exonucleases may cleave the phosphodiester bonds at the end of a polynucleotide chain at endonuclease cut sites or at ends generated by other chemical or mechanical means, such as shearing (for example by passing through fine-gauge needle, heating, sonicating, mini bead tumbling, and nebulizing), ionizing radiation, ultraviolet radiation, oxygen radicals, chemical hydrolosis and chemotherapy agents. Exonucleases may cleave the phosphodiester bonds at blunt ends or sticky ends. E. coli exonuclease I and exonuclease III are two commonly used 3′-exonucleases that have 3′-exonucleolytic single-strand degradation activity. Other examples of 3′-exonucleases include Nucleoside diphosphate kinases (NDKs), NDK1 (NM23-H1), NDK5, NDK7, and NDK8 (Yoon J-H, et al., Characterization of the 3′ to 5′ exonuclease activity found in human nucleoside diphosphate kinase 1 (NDK1) and several of its homologues. (Biochemistry 2005:44(48): 15774-15786.), WRN (Ahn, B., et al., Regulation of WRN helicase activity in human base excision repair. J. Biol. Chem. 2004, 279: 53465-53474) and Three prime repair exonuclease 2 (Trex2) (Mazur, D. J., Perrino, F. W., Excision of 3′ termini by the Trex1 and TREX2 3′→5′ exonucleases. Characterization of the recombinant proteins. J. Biol. Chem. 2001, 276: 17022-17029; both references incorporated by reference in their entireties herein). E. coli exonuclease VII and T7-exonuclease Gene 6 are two commonly used 5′-3′ exonucleases that have 5% exonucleolytic single-strand degradation activity. The exonuclease can be originated from prokaryotes, such as E. coli exonucleases, or eukaryotes, such as yeast, worm, murine, or human exonucleases. In some alternatives of the systems provided herein, the systems can further comprise an exonuclease or a vector or nucleic acid encoding an exonuclease. In some alternatives, the exonuclease is Trex2. In some alternatives of the methods provided herein, the methods can further comprise providing exonuclease or a vector or nucleic acid encoding an exonuclease, such as Trex2

“Target gene” as used herein refers to any nucleotide sequence encoding a known or putative gene product.

The term “target site” is used herein to refer to the specific locus of the target gene on a genome.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. “Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art Kyte et al, J. Mol. Biol. 157: 105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self- replicating extrachromosomal vector, and preferably, is a DNA plasmid. For example, the vector may encode an insert and/or at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular. Moreover, unless otherwise stated, the present invention was performed using standard procedures.

The present invention, as described herein, is directed to system and methods useful to modify one or more target sites on mammalian cell's genome. According to some embodiments, the system and methods described herein are useful to edit, screen, label, mark or disrupt the genome of a mammalian cell. According to some embodiments, the system and methods described herein are useful to insert genes at one or more target sites on a mammalian cell's genome.

According to some embodiments, the present invention is directed to systems and methods for modifying a target site in a mammalian cell. In some embodiments, the present invention is directed to systems and methods for modifying at least one target site in a population of mammalian cells. In some embodiments, the present invention is directed to systems and methods for modifying a plurality of target sites in a mammalian cell. In some embodiments, the present invention is directed to systems and methods for modifying a plurality of target sites in a mammalian cell population.

### Scalable Gene Insertion Systems

According to some embodiments, the present system comprises at least one type of vector and

i. At least one donor DNA construct or a sequence encoding donor DNA;

ii. At least one first guide RNA or a sequence encoding a first guide RNA;

iii. At least one second guide RNA or a sequence encoding a second guide RNA;

iv. A sequence-specific nuclease or a sequence encoding a sequence-specific nuclease.

According to some embodiments, the present system further comprises at least one additional sequence. Additional sequences may encode for additional sequence-specific nucleases, selectable markers, and/or regulatory elements that are useful for gene editing and/or expression.

### Nucleic Acid Sequences

According to some embodiments, the present invention comprises the use of one or more nucleic acid, polynucleotide, or oligonucleotide sequences, the foregoing terms being used interchangeably herein. According to some embodiments, the present nucleic acid sequences are introduced into a genome, chromosome, and etc. According to some embodiments, the present nucleic acid sequences encode for functional genes or proteins as used by the methods and systems described herein. According to some embodiments, the present nucleic acid sequences encode for the present system, components or subcomponents, such as guide RNA(s), Donor DNA(s), sequence-specific nuclease(s), selectable markers, etc., or any combination thereof. According to some embodiments, the present nucleic acid sequences encode for guide RNA. According to some embodiments, the present nucleic acid sequences encode for Donor DNA. According to some embodiments, the present nucleic acid sequences encode for a sequence-specific nuclease. The nucleic acid, poly or oligonucleotides which encode for sequences described herein may be synthesized or obtained from commercial sources. Synthesis of nucleic acid sequences is known in the art and can be by any means, including array synthesis, PCR, solid phase synthesis, or recombinant synthesis.

### Guide RNA

All of the guide RNA may not be synthesized as part of the oligonucleotide. The guide RNA may be considered as comprising a guide head and a guide tail. The guide head is about 15-22 bases in length, about 17-21 bases in length, or about 18-20 bases in length. The guide head is related in sequence to the donor DNA. The guide tail is longer and will generally be invariant in a population of plasmid constructs. The guide tail may be between about 90 and 110 bases, between about 95 and 105 bases, or between about 98 and 100 bases. The guide tail, due to its general invariance, need not be synthesized on the solid array, but can be separately synthesized by any means, including by PCR, solid phase synthesis, or recombinant synthesis. The guide tail can be joined to the oligonucleotide (containing the guide head) separately or at the same time as the oligonucleotide is joined to the plasmid.

Guide nucleic acids may be RNA or DNA molecules. They are selected and coordinated with the nucleic acid-guided sequence-specific nuclease, i.e., the properties of the guide are dictated by the sequence-specific nuclease. Many such sequence-specific nucleases are known. Guide nucleic acids are selected for complementarity to a target site of interest. Desirably the complementarity will be complete within the guide head, but for the desired mutation. Decreased complementarity may lead to loss of specificity and/or efficiency. The guide will be expressed from the plasmid in the case of a guide RNA. To achieve such expression, a suitable promoter will be placed upstream of the guide RNA-coding segment on the carrier plasmid. The transcription promoter may be synthesized as part of the oligonucleotide or may be a part of the plasmid vector. A transcription terminator may optionally be placed downstream from the guide RNA- coding segment. A terminator may prevent read-through transcription of donor nucleic acid. Any terminator functional in mammalian cells, or other desired host cells, known in the art may be used.

The present invention can include more than one different guide RNAs that target one or more different target sites, target gene or target gene specific sequences. Different guide RNAs can bind to different target genes or target gene specific sequence. According to some embodiments, the present invention comprises at least one first guide RNA, or a nucleic acid sequencing encoding a first guide RNA, at least one second guide RNA, or a nucleic acid sequencing encoding a second guide RNA.

First Guide RNA

The methods and systems described herein include at least one first guide RNA or a nucleic acid sequence encoding a first guide RNA. According to some embodiments, a first guide RNA specifically hybridizes to a target site. The first guide RNA forms a complex with an sequence-specific nuclease described herein and assists in the recognition of the intended cleavage site in the target gene or target gene specific sequence within the host cell's genome by homologous basepairing with the target gene specific sequence. In some embodiments, the first guide RNA is provided on a vector, for example, a target selector vector or gene specific vector, encoding a polynucleotide sequence for the first guide RNA.

In some embodiments, the first guide RNA targets at least one region of the target gene selected from the group consisting of a promoter region, an enhancer region, a repressor region, an insulator region, a silencer region, a region involved in DNA looping with the promoter region, a gene splicing region, or a transcribed region. In certain embodiments, the first guide RNA targets a promoter region. In certain embodiments, the first guide RNA targets an enhancer region. In certain embodiments, the first guide RNA targets a repressor region. In certain embodiments, the first guide RNA targets an insulator region. In certain embodiments, the first guide RNA targets a silencer region. In certain embodiments, the first guide RNA targets a region involved in DNA looping with the promoter region. In certain embodiments, the first guide RNA targets a gene splicing region. In certain embodiments, the first guide RNA targets a transcribed region.

According to some embodiments, the methods and systems described herein comprise a plurality of first guide RNAs or a plurality of nucleic acid sequence encoding a first guide RNA. According to some embodiments, a plurality of first guide RNAs or a plurality of nucleic acid sequence encoding a first guide RNA comprise a plurality of different first guide RNAs or a plurality of nucleic acid sequence encoding a first guide RNA, or, a plurality of same first guide RNAs or a plurality of nucleic acid sequence encoding a first guide RNA. According to some embodiment, the present invention envisions that the plurality comprises first guide RNAs or sequences encoding first guide RNAs that targets the same target site, gene, or gene specific sequence within the cell's genome. According to some embodiment, the present invention envisions that the plurality comprises first guide RNAs or sequences encoding first guide RNAs that targets different target sites within the host cell's genome.

According to some embodiments, the present invention comprises at least one to at least fifty, or more, first guide RNAs, or a nucleic acid sequencing encoding first guide RNAs. According to some embodiments, the present invention further comprises at least one to at least fifty, or more, different first guide RNAs, or a nucleic acid sequencing encoding different first guide RNAs.

Second Guide RNAs

The methods and systems described herein further include a second RNA or a nucleic acid sequence encoding a second guide RNA. According to some embodiments, a second guide RNA specifically hybridizes to donor DNA. The second guide RNA forms a complex with an sequence-specific nuclease described herein and assists in the recognition of the intended cleavage site in the target site of the sequence of interest within the donor DNA. In some embodiments, the second guide RNA is provided on a vector, for example, a donor DNA vector or donor selector vector, encoding a polynucleotide sequence for the second guide RNA. Once targeted, the can cleave the donor DNA to create the linear insert polynucleotide or a cleaved insert. The second guide RNA is specific to the donor DNA and does not target a specific sequence within the host cell's genome.

According to some embodiments, the methods and systems described herein comprise a plurality of second guide RNAs or a plurality of nucleic acid sequence encoding a second guide RNA. According to some embodiments, a plurality of second guide RNAs or a plurality of nucleic acid sequence encoding a second guide RNA comprise a plurality of different second guide RNAs or a plurality of nucleic acid sequence encoding different second guide RNAs; or, a plurality of same second guide RNAs or a plurality of nucleic acid sequence encoding a same guide RNA. According to some embodiment, the present invention envisions that the plurality comprises second guide RNAs or sequences encoding second guide RNAs that targets the same target site within the donor DNA. According to some embodiment, the present invention envisions that the plurality comprises second guide RNAs or sequences encoding second guide RNAs that targets different target sites within the donor DNA.

According to some embodiments, the present invention comprises at least one to at least fifty, or more, second guide RNAs, or a nucleic acid sequencing encoding second guide RNAs. According to some embodiments, the present invention further comprises at least one to at least fifty, or more, different second guide RNAs, or a nucleic acid sequencing encoding different second guide RNAs.

### Sequence-Specific Nucleases

The present invention further includes at least one sequence-specific nuclease or at least one nucleic acid sequence encoding at least one sequence-specific nuclease. In some embodiments, the nucleic acid-guided sequence-specific nuclease forms a complex with the 3′ end of a gRNA. The specificity of the presently described system depends on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the nucleic acid-guided sequence-specific nuclease can be directed to new genomic targets. The PAM sequence is located on the DNA to be cleaved and is recognized by a nucleic acid-guided sequence-specific nuclease. PAM recognition sequences of the nucleic acid-guided sequence-specific nuclease can be species specific.

Exemplary sequence-specific nucleases for use in the present invention include, but are not limited to, Cas, Cas9, Cas12, Clas13, AGO, PfAGO, NgAgo, TALEN, or MegaTAL. According to some embodiments, the sequence-specific nuclease is a Cas nuclease. According to some embodiments, the Cas nuclease is a Cas9 nuclease.

In some embodiments, the Cas9 nuclease is derived from a bacterial genus of Streptococcus, Staphylococcus, Brevibacillus, Corynebacter, Sutterella, Legionella, Francisella, Treponema, Filifactor, Eubacterium, Lactobacillus, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, or Campylobacter. In some embodiments, the Cas9 protein is selected from the group, including, but not limited to, Streptococcus pyogenes, Francisella novicida, Staphylococcus aureus, Neisseria meningitides, Streptococcus thermophiles, Treponema denticola, Brevibacillus laterosporus, Campylobacter jejuni, Corynebacterium diphtheria, Eubacterium ventriosum, Streptococcus pasteurianus, Lactobacillus farciminis, Sphaerochaeta globus, Azospirillum, Gluconacetobacteriazotrophicus, Neisseria cinerea, Roseburia intestinalis, Parvibaculum lavamentivorans, Nitratifractor salsuginis, and Campylobacter lari.

According to some embodiments, a Cas nuclease is a Cas9 ortholog selected from the group consisting of Streptococcus pyogenes, Staphylococcus aureus, Steptococcus thermophilus, Lactobacillus gasseri, Francisella novicida, Wolinella succinogenes, Sutterella wadsworthensis, gamma proteobacterium, Neisseria meningitidis, Camplyobacteri jejuni, Fibrobacter succinogenes, Rhodobacter speaeroides, Thermus thermophilus, Pyrococcus pyogenes, and Rhodospirillum rubrum.

In some embodiments, the Cas9 protein is selected from the group including, but not limited to, Streptococcus pyogenes Cas9 (SpCas9) endonuclease, a Francisella novicida Cas9 (FnCas9) endonuclease, a Staphylococcus aureus Cas9 (SaCas9) endonuclease, Neisseria meningitides Cas9 (NmCas9) endonuclease, Streptococcus thermophiles Cas9 (StCas9) endonuclease, Treponema denticola Cas9 (TdCas9) endonuclease, Brevibacillus laterosporus Cas9 (BlatCas9) endonuclease, Campylobacter jejuni Cas9 (CjCas9) endonuclease, a variant endonuclease thereof, or a chimera endonuclease thereof. In some embodiments, the Cas9 endonuclease is a SpCas9 variant endonuclease, a SaCas9 variant endonuclease, or a StCas9 endonuclease.

The Cas nuclease complex unwinds a DNA duplex and searches for sequences complementary to the gRNA and the correct PAM. The nuclease only mediates cleavage of the target DNA if both conditions are met. By specifying the type Cas-based nuclease and the sequence of one or more gRNA molecules, DNA cleavage sites can be localized to a specific target domain. Given that PAM sequences are variant and species specific, target sequences can be engineered to be recognized by only certain Cas9-based nucleases.

In some embodiments, the Cas9 endonuclease can recognize a PAM sequence YG, NGG, NGA, NGCG, NGAG, NGGNG, NNGRRT, NNGRRT, NNNRRT. NAAAAC, NNNNGNNT, NNAGAAW, NNNNCNDD, or NNNNRYAC.

In some embodiments, the Cas9 endonuclease is a SpCas9 endonuclease and recognizes the PAM sequence of NGG. In some embodiments, the Cas9 endonuclease is a SpCas9 variant endonuclease and recognizes the PAM sequence of NGG.

In some embodiments, the Cas9 endonuclease is a SaCas9 endonuclease and recognizes the PAM sequence of NNGRRT.

In some embodiments, the Cas9 endonuclease is a StCas9 endonuclease and recognizes the PAM sequence of NNAGAAW or NGGNG.

The present invention can include more than one different sequence-specific nuclease that target one or more different guide RNAs. Different sequence-specific nucleases can bind to different guide RNAs. According to some embodiments, the present invention comprises at least one first sequence-specific nuclease, or a nucleic acid sequencing encoding a first sequence-specific nuclease. According to some embodiments, the present invention further comprises at least one second sequence-specific nuclease, or a nucleic acid sequencing encoding a second sequence-specific nuclease. According to some embodiments, the present invention further comprises at least one additional sequence-specific nuclease, or a nucleic acid sequencing encoding an additional sequence-specific nuclease.

According to some embodiments, the present invention further comprises at least one to at least fifty, or more, additional sequence-specific nucleases, or a nucleic acid sequencing encoding additional sequence-specific nucleases. According to some embodiments, the present invention further comprises at least one to at least fifty, or more, additional different sequence-specific nucleases, or a nucleic acid sequencing encoding additional different sequence-specific nucleases.

### Donor DNA

According to some embodiments, the present invention further comprises donor DNA. As used herein, the term donor DNA may be interchangeably used with donor DNA construct. The term “construct” is as defined herein.

Like the guide RNA, the donor DNA will desirably be highly complementary to the target site on the host genome. Desirably, the only lack of complementarity will be the mutation that is introduced by the methods described herein. This may be a single nucleotide or more, in the case of insertions and deletions. The insertions and deletions may be small, e.g., 1, 2, 3, or 4 basepairs, or it may be larger, such as about 5, 10, 15, 20, 25, 30, 35, 40, 50, 60, 70, 75, 80, 100 basepairs, or more. In some embodiments, at least 18, at least 25, at least 30, at least 35, at least 40, at least 45, at least 50, at least 75, or at least 100 bp of complementarity on either side of the mutation (i.e., flanking the mutation) is desirable to achieve efficient recombination of the donor DNA into the target nucleic acid. Typically the length of the donor DNA will be about 36-200 bp, about 50-150, about 60-100 bp. Alternatively the donor may be within any range with a low end of 35, 40, 45, or 50 and a high end of 100, 150, 200, or 250 bp. If a large insertion is desired, longer overall donor lengths may be used.

According to some embodiments, the present invention further comprises at least one to at least fifty, or more, donor DNA constructs, or a nucleic acid sequencing encoding donor DNA constructs. According to some embodiments, the present invention further comprises at least one to at least fifty, or more, different donor DNA constructs, or a nucleic acid sequencing encoding different donor DNA constructs.

Donor DNA selection takes into consideration of the molecular mechanisms described herein as well as the reading frame of the target gene. The genomic open reading frame (ORF) phase regarding to the first guide RNA guided Cas9 cutting site (the last coding triplet before the cleavage loci, or, to put it another way, three base pairs upstream of the PAM sequence) is as described below, with Z representing any target genomic nucleotide A,G,C,T and “|” representing the genomic double strand break site and the surrounding nucleotides surrounding the break site numbered −2, −1, 1, 2.

ZZ₋₂ZZ₋₁|Z₁Z₂Z (Frame-0)

ZZZ₋₂Z₋₁\Z₁Z₂ (Frame-1)

ZZZ₋₂Z₋₁Z₁\Z₁ (Frame-2)

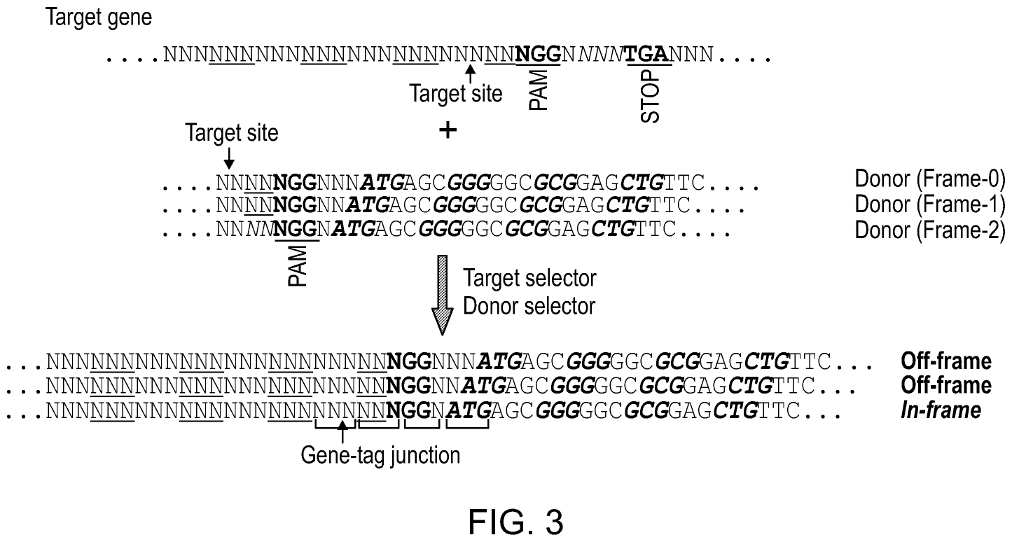

In this embodiment, the donor DNA is selected so that when cut and fused with the endogenous locus, the inserted donor product will be in frame with the upstream target gene. A representative schematic can be seen in FIG. 3. Methods of determining ORF and subsequent gene insertion are known in the art and can be seen for example in WO2019/161304, the disclosure of which is incorporated herein in its entirety.

### Mutations/Gene Insertions

The nucleic acid sequences, vectors, and cells herein may contain mutations or a collection of different mutations. For example, the donor DNA comprises a mutation. The number of different mutations represented in a library may range, for example, from 20, 25, 30, 40, 50, 100, 250, 500, 750, 1,000, 2,000, 5,000, 10,000, 100,000, or 1,000,000 to any of 100, 1,000, 10,000, 100,000, 1,000,000, 10,000,000 or 100,000,000. Ranges with any of these lower and upper limits are contemplated. Different mutations within the library may optionally code for the same amino acids, for example, when looking for optimization of translation. Alternatively, no synonymous mutations may be used within a single library. In some libraries, it may be desirable to make a mutation in every nucleotide or every codon. In other libraries it may be desirable to make all possible mutations in a codon by one or more nucleotide changes. In still other libraries it may be desirable to make mutations in a codon that lead to all possible amino acid changes.

Any type of mutation that is desirable to build into an oligonucleotide may be used. Mutations may be point mutations, deletion mutations, or insertion mutations, for example. In another example, mutations or modifications described herein may be single nucleotide polymorphism, phosphomimetic mutation, phospho null mutation, missense mutation, nonsense mutation, synonymous mutation, insertion, deletion, knock-out or knock-in. Inserted nucleic acid within an insertion mutation may be heterologous or native to the host cell.

The nucleic acid of the donor DNA may be any in which mutations are desired. The donor DNA may comprise promoter or other regulatory sequence. The donor DNA comprise a protein-coding or RNA-coding sequence. The donor DNA comprises a unknown function.

In some embodiments, mutations may be inserted into the donor DNA construct. According to some embodiments, the donor DNA construct comprises at least one mutation. According to some embodiments, the donor DNA construct comprises at least one gene insertion. According to some embodiments, the mutation comprises at least one insertion.

In some embodiments, the at least one insertion which the insertion or donor sequence is in a dual orientation to facilitate high- efficiency labeling regardless of the which orientation the insertion or mutation is inserted into the genome. In some embodiments, the present system comprises a forward copy of the first polynucleotide sequence or polynucleotide sequence encoding the insertion and a reverse copy of the first polynucleotide sequence or polynucleotide sequence encoding the insertion encoded on the same strand. In some embodiments, a polynucleotide sequence encoding a stop codon can be linked between the forward copy of the first polynucleotide sequence and a reverse copy of the first polynucleotide sequence.

In certain embodiments, at least one insertion can be incorporated at the N-terminal end of a promoter region, an enhancer region, a repressor region, an insulator region, a silencer region, a region involved in DNA looping with the promoter region, a gene splicing region, or a transcribed region thereby generating a N-terminal tagged fusion protein. In certain other embodiments, the at least one insertion is inserted at the C-terminal end of a promoter region, an enhancer region, a repressor region, an insulator region, a silencer region, a region involved in DNA looping with the promoter region, a gene splicing region, or a transcribed region thereby generating a C-terminal tagged fusion protein. In certain embodiments, the C-terminal tag can contain a Stop codon.

According to some embodiments, the insertion can encode for an antibody tag, an antibody epitope tag, a fluorescent protein, an affinity purification tag, a protein-protein interaction domain, a chemically induced protein-protein interaction domain, an enzyme, a recombinase site, a protein stability regulating tag, a spatial localization sequence, DNA/RNA targeting proteins, or any combination thereof.

Examples of antibody tags include human influenze hemagglutinin (HA), and the like.

Examples of antibody epitope tags include an Myc tag, a VS tag, and the like.

Examples of an affinity purification tag includes a Biotin tag, a His tag, and the like.

Examples of a fluorescent protein tag includes GFP, YFP, RFP, mNeonGreen, TdTomato, and the like.

As used herein, the term “fluorescent protein” refers to a protein domain that comprises at least one organic compound moiety that emits fluorescent light in response to the appropriate wavelengths. For example, fluorescent proteins may emit red, blue and/or green light. Such proteins are readily commercially available including, but not limited to: i) mCherry (Clonetech Laboratories): excitation: 556/20 nm (wavelength/bandwidth); emission: 630/91 nm; ii) sfGFP (Invitrogen): excitation: 470/28 nm; emission: 512/23 nm; iii) TagBFP (Evrogen): excitation 387/11 nm; emission 464/23 nm.

Examples of a protein-protein interaction domain include a leucine zipper, PDZ domain, and the like.

Examples of a chemically induced protein-protein interaction domain include FKBP-FRB; ABI-PYL; GAI-GID, and the like.

Examples of an enzyme include BirA, ascorbate peroxidase, and the like.

Examples of a recombinase site include loxP, FRT, Vlox and the like.

Examples of a protein stability regulating tag include chemically stabilized FKBP variants, PEST domain, and the like.

Examples of a spatial localization sequence include ER-retention sequence, nuclear localization signal, plasma membrane localization sequence, and the like.

Examples of DNA/RNA targeting proteins, include Cas9, Cas12, Cas13, and the like.

The present invention can include more than one different gene insertions that when transcribed express one or more different proteins. Different gene insertions transcribe to different proteins. According to some embodiments, the present invention further comprises at least one to at least fifty, or more, gene insertions constructs, or a nucleic acid sequencing encoding gene insertions. According to some embodiments, the present invention further comprises at least one to at least fifty, or more, different gene insertions, or a nucleic acid sequencing encoding different gene insertions.

According to some embodiments, if more than donor DNA is used then a heterologous cell population may be obtained. According to some embodiments, if more than one donor DNA is used then an isogeneic cell population may be obtained. According to some embodiments, if more than one donor DNA is used then a cell population with heterologous protein expression may be obtained. According to some embodiments, if more than one donor DNA is used then a cell population with homologous protein expression may be obtained.

According to some embodiments, if more than one gene insertion is used then a heterologous cell population may be obtained. According to some embodiments, if more than one gene insertion is used then an isogeneic cell population may be obtained. According to some embodiments, if more than one gene insertion is used then a cell population with heterologous protein expression may be obtained. According to some embodiments, if more than one gene insertion is used then a cell population with homologous protein expression may be obtained.

According to some embodiments, if more than one fluorescent protein tag is used then a heterologous cell population may be obtained. According to some embodiments, if more than one fluorescent protein tag is used then an isogeneic cell population may be obtained. According to some embodiments, if more than one fluorescent protein tag is used then a cell population with heterologous protein expression may be obtained. According to some embodiments, if more than one fluorescent protein tag is used then a cell population with homologous protein expression may be obtained. For example, if more than one fluorescent tag is used then a cell population expressing more than one fluorescent tag may be obtained emitting more than one color, emitting more than one wavelength, or both.

### Selectable Markers

According to some embodiments, the present invention further comprises a selectable marker. According to some embodiments, the selectable marker is a gene insertion. According to some embodiments, the selection marker is additional to a gene insertion.

As used herein, “selectable marker” means a nucleotide sequence that when expressed imparts a distinct phenotype to the host cell expressing the marker and thus allows such transformed cells to be distinguished from those that do not have the marker. Such a nucleotide sequence can encode either a selectable or screenable marker, depending on whether the marker confers a trait that can be selected for by chemical means, such as by using a selective agent (e.g., an antibiotic and the like), or whether the marker is simply a trait that one can identify through observation or testing, such as by screening (e.g., fluorescence). Of course, many examples of suitable selectable markers are known in the art and can be used in the vectors described herein.

### Vectors

The genetic construct, such as a plasmid, expression cassette or vector, can comprise nucleic acids that encodes the systems, components, or subcomponents described herein, for example, first guide RNAs, second guide RNAs, sequence-specific nucleases, and donor DNAs. The nucleic acid sequences can make up a genetic construct that can be a vector wherein the vector is capable of expressing the system, components or subcomponents described herein in the cell of a mammal.

The term “vector” refers to a composition for transferring, delivering or introducing a nucleic acid (or nucleic acids) into a cell. A vector comprises a nucleic acid molecule comprising the nucleotide sequencers) to be transferred, delivered or introduced. Vectors for use in transformation of host organisms are well known in the art Non-limiting examples of general classes of vectors include but are not limited to a viral vector, a plasmid vector, a phage vector, a phagemid vector, a cosmid vector, a fosmid vector, a shuttle vector, a bacteriophage, an artificial chromosome, or an Agrobacterium binary vector in double or single stranded linear or circular form which may or may not be self transmissible or mobilizable. A vector as defined herein can transform a eukaryotic host either by integration into the cellular genome or exist as an extrachromosomal element (e.g., minichromosome).

According to some embodiments of the disclosure, the genetic constructs and polynucleotides comprising polynucleotides encoding the first guide RNAs sequences, second guide RNAs sequences, Donor DNAs sequences, and sequence-specific nucleases sequences can be operatively associated with a variety of promoters, terminators and other regulatory elements for expression in various organisms or cells. In some embodiments, the genetic constructs can comprise regulatory elements for gene expression of the coding sequences of the nucleic acid. In some embodiments, the regulatory elements can be a promoter, an enhancer, an initiation codon, a stop codon, or a polyadenylation signal.

According to some embodiments, at least one promoter and/or terminator, such as a poly A signal, can be operably linked to a polynucleotide of the disclosure. Any promoter useful with this disclosure can be used and includes, for example, promoters functional with the organism of interest including but not limited to constitutive, inducible, developmentally regulated, and the like, as described herein.

In some aspects, the polynucleotide, or polynucleotides encoding the present system that is introduced into a eukaryotic cell are operably linked to a promoter and/or to a poly A signal as known in the art. Therefore, in some aspects, the nucleic acid constructs of the disclosure encoding the polypeptides of the present system having a 5′ end and a 3′ end can be operably linked at the 5′ end to a promoter and at the 3′ end to a poly A signal. In some aspects, the nucleic acid constructs of the disclosure can comprise 2A peptide sequences and/or internal ribosomal entry sites as known in the art for assisting the co-translation of multiple independent polypeptides (proteins). In some aspects, the nucleic acid constructs of the disclosure encoding the polypeptides or proteins of the present system can be introduced into a eukaryotic cell via a plasmid, a viral vector, or a nanoparticle.

The vectors described herein also can optionally include a transcriptional and/or translational termination region (i.e., termination region) that is functional in the selected host cell. A variety of transcriptional terminators is available for use in the present vectors and can be responsible for the termination of transcription beyond the heterologous nucleotide sequence of interest. The termination region can be native to the transcriptional initiation region, can be native to the operably linked nucleotide sequence of interest, can be native to the host cell, or can be derived from another source (i.e., foreign or heterologous to the promoter, to the nucleotide sequence of interest, to the host, or any combination thereof). In some embodiments of this disclosure, terminators can be operably linked to a recombinant polynucleotides) encoding the present system or subcomponents thereof.

The vectors can also can include a nucleotide sequence encoding a selectable marker, which can be used to select a transformed host cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence can encode either a selectable or screenable marker, depending on whether the marker confers a trait that can be selected for by chemical means, such as by using a selective agent (e.g., an antibiotic and the like), or whether the marker is simply a trait that one can identify through observation or testing, such as by screening (e.g., fluorescence). Of course, many examples of suitable selectable markers are known in the art and can be used in the vectors described herein.

Coding sequences can be optimized for stability and high levels of expression. In some instances, codons are selected to reduce secondary structure formation of the RNA such as that formed due to intramolecular bonding.

According to some embodiments, the reading frame of the coding sequences, constructs, vectors, or any combination thereof are optimized for appropriate expression.

In some embodiments, the promoter can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Ban- virus (EBV) promoter, a U6 promoter, such as the human U6 promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. In some embodiments, the promoter is a type III RNA polymerase III promoter. In some embodiments, the promoter is a U6 promoter, a HI promoter, or a 7SK promoter.

The polyadenylation signal can be a SV40 polyadenylation signal, LTR polyadenylation signal, bovine growth hormone (bGH) polyadenylation signal, human growth hormone (hGH) polyadenylation signal, or human β- globin polyadenylation signal. The SV40 polyadenylation signal can be a polyadenylation signal from a pCEP4 vector (Invitrogen, San Diego, Calif.).

The enhancer can be necessary for DNA expression. The enhancer can be human actin, human myosin, human hemoglobin, human muscle creatine or a viral enhancer such as one from CMV, HA, RSV or EBV. Polynucleotide function enhancers are described in U.S. Pat. Nos. 5,593,972, 5,962,428, and WO94/016737, the contents of each are fully incorporated by reference. The vector can also comprise a mammalian origin of replication in order to maintain the vector extrachromosomally and produce multiple copies of the vector in a cell. The vector can also comprise a regulatory sequence, which can be well suited for gene expression in a mammalian or human cell into which the vector is administered. The vector can also comprise a reporter gene, such as green fluorescent protein (“GFP”), yellow fluorescent protein (“YFP”), or red fluorescent protein (“RFP”), and/or a selectable marker, such as puromycin (“PuroP”), blasticidin (“bsr”), or nourseothricin (“NTC”).

In some embodiments, the polynucleotide or genetic construct encoding the present system, or subcomponents thereof, can be introduced in one construct or in different constructs. In some embodiments, the genetic constructs can be located in a single vector or included on multiple different vectors.

The systems and methods described herein can include one or more vectors wherein a vector can include one or more polynucleotide sequences, such as one or more first polynucleotide sequences, one or more second polynucleotide sequences, one or more third polynucleotide sequences, one or more fourth polynucleotide sequences, and one or more additional polynucleotide sequences.

In some embodiments, one or more first polynucleotide sequences can encode at least 1 first guide RNA. For example, the one or more first polynucleotide sequences can encode at least 1 first guide RNA, at least 2 first guide RNAs, at least 3 first guide RNAs, at least 4 first guide RNAs, at least 5 first guide RNAs, at least 6 first guide RNAs, at least 7 first guide RNAs, at least 8 first guide RNAs, at least 9 first guide RNAs, at least 10 first guide RNA, at least 11 first guide RNA, at least 12 first guide RNAs, at least 13 first guide RNAs, at least 14 first guide RNAs, at least 15 first guide RNAs, at least 16 first guide RNA, at least 17 first guide RNAs, at least 18 first guide RNAs, at least 19 first guide RNAs, at least 20 first guide RNAs, at least 25 first guide RNA, at least 30 first guide RNAs, at least 35 first guide RNAs, at least 40 first guide RNAs, at least 45 first guide RNAs, or at least 50 first guide RNAs.

In some embodiments, the one or more first polynucleotide sequences can encode between 1 first guide RNA and 50 first guide RNAs, between 1 first guide RNA and 45 first guide RNAs, between 1 first guide RNA and 40 first guide RNAs, between 1 first guide RNA and 35 first guide RNAs, between 1 first guide RNA and 30 first guide RNAs, between 1 first guide RNA and 25 different first guide RNAs, between 1 first guide RNA and 20 first guide RNAs, between 1 first guide RNA and 16 first guide RNAs, between 1 first guide RNA and 8 different first guide RNAs, between 4 different first guide RNAs and 50 different first guide RNAs, between 4 different first guide RNAs and 45 different first guide RNAs, between 4 different first guide RNAs and 40 different first guide RNAs, between 4 different first guide RNAs and 35 different first guide RNAs, between 4 different first guide RNAs and 30 different first guide RNAs, between 4 different first guide RNAs and 25 different first guide RNAs, between 4 different first guide RNAs and 20 different first guide RNAs, between 4 different first guide RNAs and 16 different first guide RNAs, between 4 different first guide RNAs and 8 different first guide RNAs, between 8 different first guide RNAs and 50 different first guide RNAs, between 8 different first guide RNAs and 45 different first guide RNAs, between 8 different first guide RNAs and 40 different first guide RNAs, between 8 different first guide RNAs and 35 different first guide RNAs, between 8 different first guide RNAs and 30 different first guide RNAs, between 8 different first guide RNAs and 25 different first guide RNAs, between 8 different first guide RNAs and 20 different first guide RNAs, between 8 different first guide RNAs and 16 different first guide RNAs, between 16 different first guide RNAs and 50 different first guide RNAs, between 16 different first guide RNAs and 45 different first guide RNAs, between 16 different first guide RNAs and 40 different first guide RNAs, between 16 different first guide RNAs and 35 different first guide RNAs, between 16 different first guide RNAs and 30 different first guide RNAs, between 16 different first guide RNAs and 25 different first guide RNAs, or between 16 different first guide RNAs and 20 different first guide RNAs, or more. In some embodiments, each of the polynucleotide sequences encoding the different first guide RNAs can be operably linked to a promoter. In some embodiments, the promoters that are operably linked to the different first guide RNAs can be the same promoter. In some embodiments, the promoters that are operably linked to the different first guide RNAs can be different promoters. The promoter can be a constitutive promoter, an inducible promoter, a repressible promoter, or a regulatable promoter. At least one first guide RNA can bind to a target gene or loci of the host genome. If more than one first guide RNA is included, each of the first guide RNAs binds to a different target region within one target loci or each of the first guide RNA binds to a different target region within different gene loci.

According to some embodiments, the present invention further comprises one or more second polynucleotide sequences. In some embodiments, the one or more second polynucleotide sequence can encode at least 1 second guide RNA. For example, the one or more second polynucleotide sequences can encode at least 1 second guide RNA, at least 2 second guide RNAs, at least 3 second guide RNAs, at least 4 second guide RNAs, at least 5 second guide RNAs, at least 6 second guide RNAs, at least 7 second guide RNAs, at least 8 second guide RNAs, at least 9 second guide RNAs, at least 10 second guide RNA, at least 11 second guide RNA, at least 12 second guide RNAs, at least 13 second guide RNAs, at least 14 second guide RNAs, at least 15 second guide RNAs, at least 16 second guide RNA, at least 17 second guide RNAs, at least 18 second guide RNAs, at least 19 second guide RNAs, at least 20 second guide RNAs, at least 25 second guide RNA, at least 30 second guide RNAs, at least 35 second guide RNAs, at least 40 second guide RNAs, at least 45 second guide RNAs, or at least 50 second guide RNAs.

In some embodiments, the one or more second polynucleotide sequences can encode between 1 second guide RNA and 50 second guide RNAs, between 1 second guide RNA and 45 second guide RNAs, between 1 second guide RNA and 40 second guide RNAs, between 1 second guide RNA and 35 second guide RNAs, between 1 second guide RNA and 30 second guide RNAs, between 1 second guide RNA and 25 different second guide RNAs, between 1 second guide RNA and 20 second guide RNAs, between 1 second guide RNA and 16 second guide RNAs, between 1 second guide RNA and 8 different second guide RNAs, between 4 different second guide RNAs and 50 different second guide RNAs, between 4 different second guide RNAs and 45 different second guide RNAs, between 4 different second guide RNAs and 40 different second guide RNAs, between 4 different second guide RNAs and 35 different second guide RNAs, between 4 different second guide RNAs and 30 different second guide RNAs, between 4 different second guide RNAs and 25 different second guide RNAs, between 4 different second guide RNAs and 20 different second guide RNAs, between 4 different second guide RNAs and 16 different second guide RNAs, between 4 different second guide RNAs and 8 different second guide RNAs, between 8 different second guide RNAs and 50 different second guide RNAs, between 8 different second guide RNAs and 45 different second guide RNAs, between 8 different second guide RNAs and 40 different second guide RNAs, between 8 different second guide RNAs and 35 different second guide RNAs, between 8 different second guide RNAs and 30 different second guide RNAs, between 8 different second guide RNAs and 25 different second guide RNAs, between 8 different second guide RNAs and 20 different second guide RNAs, between 8 different second guide RNAs and 16 different second guide RNAs, between 16 different second guide RNAs and 50 different second guide RNAs, between 16 different second guide RNAs and 45 different second guide RNAs, between 16 different second guide RNAs and 40 different second guide RNAs, between 16 different second guide RNAs and 35 different second guide RNAs, between 16 different second guide RNAs and 30 different second guide RNAs, between 16 different second guide RNAs and 25 different second guide RNAs, or between 16 different second guide RNAs and 20 different second guide RNAs, or more. In some embodiments, each of the polynucleotide sequences encoding the different second guide RNAs can be operably linked to a promoter. In some embodiments, the promoters that are operably linked to the different second guide RNAs can be the same promoter. In some embodiments, the promoters that are operably linked to the different second guide RNAs can be different promoters. The promoter can be a constitutive promoter, an inducible promoter, a repressible promoter, or a regulatable promoter. At least one second guide RNA can bind to a target gene or loci of the donor DNA. If more than one second guide RNA is included, each of the second guide RNAs binds to a different target region within one target loci or each of the second guide RNA binds to a different target region within different gene loci.

According to some embodiments, the present invention further comprises one or more third or fourth polynucleotide sequences. According to some embodiments, the one or more third or fourth polynucleotide sequences can encode at least 1 sequence-specific nuclease. For example, the one or more third or fourth polynucleotide sequences can encode at least 1 sequence-specific nuclease, at least 2 sequence-specific nucleases, at least 3 sequence-specific nucleases, at least 4 sequence-specific nucleases, at least 5 sequence-specific nucleases, at least 6 sequence-specific nucleases, at least 7 sequence-specific nucleases, at least 8 sequence-specific nucleases, at least 9 sequence-specific nucleases, at least 10 sequence-specific nuclease, at least 11 sequence-specific nuclease, at least 12 sequence-specific nucleases, at least 13 sequence-specific nucleases, at least 14 sequence-specific nucleases, at least 15 sequence-specific nucleases, at least 16 sequence-specific nuclease, at least 17 sequence-specific nucleases, at least 18 sequence-specific nucleases, at least 19 sequence-specific nucleases, at least 20 sequence-specific nucleases, at least 25 sequence-specific nuclease, at least 30 sequence-specific nucleases, at least 35 sequence-specific nucleases, at least 40 sequence-specific nucleases, at least 45 sequence-specific nucleases, or at least 50 sequence-specific nucleases.

In some embodiments, the one or more third or fourth polynucleotide sequences can encode between 1 sequence-specific nuclease and 50 sequence-specific nucleases, between 1 sequence-specific nuclease and 45 sequence-specific nucleases, between 1 sequence-specific nuclease and 40 sequence-specific nucleases, between 1 sequence-specific nuclease and 35 sequence-specific nucleases, between 1 sequence-specific nuclease and 30 sequence-specific nucleases, between 1 sequence-specific nuclease and 25 different sequence-specific nucleases, between 1 sequence-specific nuclease and 20 sequence-specific nucleases, between 1 sequence-specific nuclease and 16 sequence-specific nucleases, between 1 sequence-specific nuclease and 8 different sequence-specific nucleases, between 4 different sequence-specific nucleases and 50 different sequence-specific nucleases, between 4 different sequence-specific nucleases and 45 different sequence-specific nucleases, between 4 different sequence-specific nucleases and 40 different sequence-specific nucleases, between 4 different sequence-specific nucleases and 35 different sequence-specific nucleases, between 4 different sequence-specific nucleases and 30 different sequence-specific nucleases, between 4 different sequence-specific nucleases and 25 different sequence-specific nucleases, between 4 different sequence-specific nucleases and 20 different sequence-specific nucleases, between 4 different sequence-specific nucleases and 16 different sequence-specific nucleases, between 4 different sequence-specific nucleases and 8 different sequence-specific nucleases, between 8 different sequence-specific nucleases and 50 different sequence-specific nucleases, between 8 different sequence-specific nucleases and 45 different sequence-specific nucleases, between 8 different sequence-specific nucleases and 40 different sequence-specific nucleases, between 8 different sequence-specific nucleases and 35 different sequence-specific nucleases, between 8 different sequence-specific nucleases and 30 different sequence-specific nucleases, between 8 different sequence-specific nucleases and 25 different sequence-specific nucleases, between 8 different sequence-specific nucleases and 20 different sequence-specific nucleases, between 8 different sequence-specific nucleases and 16 different sequence-specific nucleases, between 16 different sequence-specific nucleases and 50 different sequence-specific nucleases, between 16 different sequence-specific nucleases and 45 different sequence-specific nucleases, between 16 different sequence-specific nucleases and 40 different sequence-specific nucleases, between 16 different sequence-specific nucleases and 35 different sequence-specific nucleases, between 16 different sequence-specific nucleases and 30 different sequence-specific nucleases, between 16 different sequence-specific nucleases and 25 different sequence-specific nucleases, or between 16 different sequence-specific nucleases and 20 different sequence-specific nucleases, or more. In some embodiments, each of the polynucleotide sequences encoding the different sequence-specific nucleases can be operably linked to a promoter. In some embodiments, the promoters that are operably linked to the different sequence-specific nucleases can be the same promoter. In some embodiments, the promoters that are operably linked to the different sequence-specific nucleases can be different promoters. The promoter can be a constitutive promoter, an inducible promoter, a repressible promoter, or a regulatable promoter. At least one sequence-specific nuclease can complex with a gRNA. If more than one sequence-specific nuclease is included, each of the sequence-specific nuclease binds to a different gRNA.

According to some embodiments, the present invention further comprises one or more additional sequences. In some embodiments, the one or more additional polynucleotide sequence can encode at least 1 donor DNA construct. For example, the one or more additional polynucleotide sequences can encode at least 1 donor DNA construct, at least 2 donor DNA constructs, at least 3 donor DNA constructs, at least 4 donor DNA constructs, at least 5 donor DNA constructs, at least 6 donor DNA constructs, at least 7 donor DNA constructs, at least 8 donor DNA constructs, at least 9 donor DNA constructs, at least 10 donor DNA construct, at least 11 donor DNA construct, at least 12 donor DNA constructs, at least 13 donor DNA constructs, at least 14 donor DNA constructs, at least 15 donor DNA constructs, at least 16 donor DNA construct, at least 17 donor DNA constructs, at least 18 donor DNA constructs, at least 19 donor DNA constructs, at least 20 donor DNA constructs, at least 25 donor DNA construct, at least 30 donor DNA constructs, at least 35 donor DNA constructs, at least 40 donor DNA constructs, at least 45 donor DNA constructs, or at least 50 donor DNA constructs.

In some embodiments, the one or more additional polynucleotide sequences can encode between 1 donor DNA construct and 50 donor DNA constructs, between 1 donor DNA construct and 45 donor DNA constructs, between 1 donor DNA construct and 40 donor DNA constructs, between 1 donor DNA construct and 35 donor DNA constructs, between 1 donor DNA construct and 30 donor DNA constructs, between 1 donor DNA construct and 25 different donor DNA constructs, between 1 donor DNA construct and 20 donor DNA constructs, between 1 donor DNA construct and 16 donor DNA constructs, between 1 donor DNA construct and 8 different donor DNA constructs, between 4 different donor DNA constructs and 50 different donor DNA constructs, between 4 different donor DNA constructs and 45 different donor DNA constructs, between 4 different donor DNA constructs and 40 different donor DNA constructs, between 4 different donor DNA constructs and 35 different donor DNA constructs, between 4 different donor DNA constructs and 30 different donor DNA constructs, between 4 different donor DNA constructs and 25 different donor DNA constructs, between 4 different donor DNA constructs and 20 different donor DNA constructs, between 4 different donor DNA constructs and 16 different donor DNA constructs, between 4 different donor DNA constructs and 8 different donor DNA constructs, between 8 different donor DNA constructs and 50 different donor DNA constructs, between 8 different donor DNA constructs and 45 different donor DNA constructs, between 8 different donor DNA constructs and 40 different donor DNA constructs, between 8 different donor DNA constructs and 35 different donor DNA constructs, between 8 different donor DNA constructs and 30 different donor DNA constructs, between 8 different donor DNA constructs and 25 different donor DNA constructs, between 8 different donor DNA constructs and 20 different donor DNA constructs, between 8 different donor DNA constructs and 16 different donor DNA constructs, between 16 different donor DNA constructs and 50 different donor DNA constructs, between 16 different donor DNA constructs and 45 different donor DNA constructs, between 16 different donor DNA constructs and 40 different donor DNA constructs, between 16 different donor DNA constructs and 35 different donor DNA constructs, between 16 different donor DNA constructs and 30 different donor DNA constructs, between 16 different donor DNA constructs and 25 different donor DNA constructs, or between 16 different donor DNA constructs and 20 different donor DNA constructs, or more. In some embodiments, each of the polynucleotide sequences encoding the different donor DNA constructs can be operably linked to a promoter. In some embodiments, the promoters that are operably linked to the different donor DNA constructs can be the same promoter. In some embodiments, the promoters that are operably linked to the different donor DNA constructs can be different promoters. The promoter can be a constitutive promoter, an inducible promoter, a repressible promoter, or a regulatable promoter. At least one donor DNA construct can hybridize with a target site on the host genome. If more than one donor DNA construct is included, each of the donor DNA construct binds to a different target site.

According to some embodiments, the present invention further comprises one or more additional sequences. In some embodiments, the one or more additional polynucleotide sequences can encode at least 1 selectable maker. For example, the one or more additional polynucleotide sequences can encode at least 1 selectable maker, at least 2 selectable makers, at least 3 selectable makers, at least 4 selectable makers, at least 5 selectable makers, at least 6 selectable makers, at least 7 selectable markers, at least 8 selectable makers, at least 9 selectable makers, at least 10 selectable maker, at least 11 selectable maker, at least 12 selectable makers, at least 13 selectable makers, at least 14 selectable markers, at least 15 selectable makers, at least 16 selectable maker, at least 17 selectable markers, at least 18 selectable makers, at least 19 selectable makers, at least 20 selectable makers, at least 25 selectable maker, at least 30 selectable makers, at least 35 selectable makers, at least 40 selectable markers, at least 45 selectable makers, or at least 50 selectable makers.

In some embodiments, the one or more additional polynucleotide sequences can encode between 1 selectable maker and 50 selectable makers, between 1 selectable maker and 45 selectable markers, between 1 selectable maker and 40 selectable makers, between 1 selectable maker and 35 selectable markers, between 1 selectable maker and 30 selectable makers, between 1 selectable maker and 25 different selectable markers, between 1 selectable maker and 20 selectable makers, between 1 selectable marker and 16 selectable makers, between 1 selectable maker and 8 different selectable markers, between 4 different selectable markers and 50 different selectable markers, between 4 different selectable markers and 45 different selectable markers, between 4 different selectable markers and 40 different selectable markers, between 4 different selectable markers and 35 different selectable markers, between 4 different selectable markers and 30 different selectable markers, between 4 different selectable markers and 25 different selectable markers, between 4 different selectable markers and 20 different selectable markers, between 4 different selectable markers and 16 different selectable markers, between 4 different selectable markers and 8 different selectable markers, between 8 different selectable markers and 50 different selectable markers, between 8 different selectable markers and 45 different selectable markers, between 8 different selectable markers and 40 different selectable markers, between 8 different selectable markers and 35 different selectable markers, between 8 different selectable markers and 30 different selectable markers, between 8 different selectable markers and 25 different selectable markers, between 8 different selectable markers and 20 different selectable markers, between 1 different selectable markers and 16 different selectable markers, between 16 different selectable markers and 50 different selectable markers, between 16 different selectable markers and 45 different selectable markers, between 16 different selectable markers and 40 different selectable markers, between 16 different selectable markers and 35 different selectable markers, between 16 different selectable markers and 30 different selectable markers, between 16 different selectable markers and 25 different selectable markers, or between 16 different selectable markers and 20 different selectable markers, or more. In some embodiments, each of the polynucleotide sequences encoding the different selectable markers can be operably linked to a promoter. In some embodiments, the promoters that are operably linked to the different selectable markers can be the same promoter. In some embodiments, the promoters that are operably linked to the different selectable markers can be different promoters. The promoter can be a constitutive promoter, an inducible promoter, a repressible promoter, or a regulatable promoter. At least one selectable marker when transcribed expresses a phenotypic protein. If more than one selectable marker is included, each of the selectable marker expresses a different phenotypic protein.

In some embodiments, one or more of the genetic constructs described herein may be packaged in one or more plasmid vectors. For example, the first guide RNAs, second guide RNAs, Donor DNAs, and sequence-specific nucleases in the same or different plasmid vectors.

In some embodiments, one or more of the genetic constructs described herein may be packaged in a viral vector. For example, the first guide RNAs may be packaged in a viral vector, for example a lentivirus vector.

Plasmid Vector

The vector can be a plasmid. The vector can be useful for transfecting cells with nucleic acid encoding the first guide RNAs, second guide RNAs, Donor DNAs, and sequence-specific nucleases described herein, which the transformed host cell is cultured and maintained under conditions wherein expression of the genetic insert takes place. Plasmids which can be used in the methods described include any that have an origin of replication that is functional in the target cells. These plasmids will typically be linearizable. Often such linearization will be accomplished with a restriction endonuclease that cleaves the plasmid one or a few times only. Other methods, enzymatic or mechanical can be used for linearization. Often the plasmid will have one or more markers that are selectable or easily screenable in an intermediate host cells and/or in the target cells. For example, an antibiotic resistance gene can be used for selecting in a host cell, such as puromycin, blasticidin, or nourothricin. Transcription regulatory elements such as promoters and terminators may also be in the plasmid for controlling transcription of elements of the oligonucleotide.

The systems described herein can include a plasmid vector. The plasmid vector can include one or more polynucleotide sequences encoding first guide RNAs, second guide RNAs, sequence-specific nucleases, or donor DNAs, alone, together, or any combination thereof. The plasmid vector comprises at least one or more of a first, second, third, fourth, etc. polynucleotide sequences encoding a component of the system described herein, such as first guide RNAs, second guide RNAs, sequence-specific nucleases, or subcomponents thereof.

Lentiviral Vector

Lentiviral vector is a vector belonging to the lentivirus family of retroviruses that are able to infect human and other mammalian species. The systems described herein can include a modified lentiviral vector. The modified lentiviral vector can include one or more polynucleotide sequences encoding first guide RNAs, second guide RNAs, sequence-specific nucleases, or donor DNAs, alone, together, or any combination thereof. The modified lentiviral vector comprises a first, second, third, fourth, etc. polynucleotide sequences encoding at least one component of the system described herein, such as first guide RNAs, second guide RNAs, sequence-specific nucleases, or subcomponents thereof. The one or more polynucleotide sequences can be operably linked to a eukaryotic promoter. The promoter can be a constitutive promoter, an inducible promoter, a repressible promoter, or a regulatable promoter.

Delivery

The genetic constructs disclosed in the present invention may be delivered using any method of DNA delivery to cells, including non- viral and viral methods. Common non-viral delivery methods include transformation and transfection. Non-viral gene delivery can be mediated by physical methods such as electroporation, microinjection, particle-medicated gene transfer (‘gene gun’), impalefection, hydrostatic pressure, continuous infusion, sonication, chemical transfection, lipofection, or DNA injection (DNA vaccination) with and without in vivo electroporation. Viral mediated gene delivery, or viral transduction, utilizes the ability of a virus to inject its DNA inside a host cell. In some embodiments, the genetic constructs intended for delivery are packaged into a replication-deficient viral particle. Common viruses used include retrovirus, lentivirus, adenovirus, adeno-associated virus, and herpes simplex virus.

### Host Cells

Host cells which can be used are any mammalian cells that can be transformed with nucleic acids or otherwise made to efficiently take up nucleic acids. The host cells may be those that naturally make useful products or those that are engineered to make useful products.

According to some embodiments, the present system and methods can be used with any mammalian cell line, including known cancer lines (for example, hela, MCF7, or K562), primary cells (patient fibroblasts), stem cells (induced pluripotent stem cells and embryonic stem cells), organoids, or any other commonly used cell culture system. In some embodiments, the host cell is selected from the group including, but not limited to, a myoblast, a fibroblast, a glioblastoma, a carcinoma, an epithelial cell, a stem cell. In some embodiments, the host cell is selected from the group including, but not limited to, a HEK cell, a HeLa cell, a vero cell, a BHK cell, a MDCK cell, a NIH 3T3 cell, a Neuro-2a cell, and a CHO cell.

A wide variety of cell lines suitable for use as a host cell include, but are not limited to, C8161I, CCRF-CEM, MOLT, mIMCD-3, NHDF, HeLa˜S3, Huh1, Huh4, Huii7, HUVEC, HASMC, HEKn, HEKa, MiaPaCell, Panel, PC-3, TF1, CTLL-2, CIR, Rat6, CV1, RPTE, A10, T24, .182, A375, ARH-77, Calul, SW480, SW620, S OV3, S -UT, CaCo2, P388D1, SEM-K2, WEHI-231, HB56, TIB55, Jurkat, J45.0L LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A.?.780, A2780ADR, A2780cis, A172, A20, A253, A431, A-549, ALC, B16, B35, BCP-1 cells, BEAS- 2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR- L23/R23, COS-7, COV-434, CML TL CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepal c1 c7, HL-60, HMEC, HT-29, Jurkat, JY cells, 562 cells, Ku812, KCL22, G 1, KY01, LNCap, Via- ic! 1-48, MC-38, MCF-7, MCF-IOA, MDA-MB-231, MDA-MB-468, MDA-MB- 435, MDCK II, MDCK 11, MOR/0.2R, MONO-MAC 6, MTD-1 A, MyEnd, NCI-H69/CPR, NCI-H69/LX10, NCI-H69/LX20, NQ-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vera cells, WM39, WT-49, X63, YAC-1, YAR, and transgenic varieties thereof. Cell lines are available from a variety of sources known to those with skill in the art (see, e.g., the American Type Culture Collection (ATCC) (Manassus, Va.)).

Any means of transformation of cells can be used. However, high efficiency of transformation is desirable. One feature that contributes to high efficiency is the linkage of nucleic acid encoding guide nucleic acid to nucleic acid encoding donor nucleic acid. Such linkage insures that any transformed cell has both components necessary for making a mutation in the host cell. Another feature that may contribute to high efficiency of transformation of cells is the use of DMSO, for example, DMSO at a final concentration of 10%, prior to a step of heat shock. Other amounts of DMSO may be used for example 5, 6, 7, 8, 9, 11, 12, 13, 14, 15, or 16 percent.

In general, any technique known in the art for eukaryotic, such as mammalian, cell transformation can be used, including but not limited to use of lithium, electroporation, biolistic and glass bead methods. Transformation efficiencies, depending on the host cell, and the particular elements used in the transforming nucleic acids, may vary, for example, from at least 20, at least 25, at least 30, at least 35, at least 40, at least 45, at least 50, at least 60, at least 70, at least 80, at least 90, or at least 95% up to 100%.

Mammalian cells used as recipients of the carrier plasmid library may express a nucleic acid-guided endonuclease. Typically this will be a heterologous enzyme that the yeast has been previously engineered to express. Any of the nucleic acid-guided endonucleases may be used. Any technique known in the art for introducing and expressing a nucleic acid encoding the endonuclease may be used. Alternately, a nucleic acid encoding the endonuclease may be introduced with the plasmid into the mammalian cell. Another option is to introduce the endonuclease after addition of the plasmid library. Selection among such options is within the skill of the art.

Examples of useful mammalian cells include human cells, for example, HEK blue-mCherry-CAS9 cells (HEK 293 cells) or HEK 293T cells.

According to some embodiments, target genes in the host cell may include TUBB3 gene, MAP2 gene, MECP2 gene, NRCAM gene, ACTR2 gene, CLTA gene, ANK3 gene, SPTBN4 gene, SCN2A gene, GFAP gene, PDHA 1 gene, or DOT gene. In some embodiments, the target gene can be TUBB5 gene, INSYN1 gene, INSYN2 gene, ARHGAP32 gene, TUBB gene, ACTB gene, IMNB1 gene, or NEFM gene.

### Host Cell Integration

Methods of introducing a nucleic acid into a host cell are known in the art, and any known method can be used to introduce a nucleic acid (e.g., an expression construct) into a cell. Suitable methods include, include e.g., viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome- mediated transfection, particle gun technology, calcium phosphate precipitation, direct micro injection, nanoparticle-mediated nucleic acid delivery, and the like.

### Selection

Transformed mammalian cells can be recovered (i.e., isolated and purified) by selection for a marker on the vector, such as an antibiotic resistance markers. Selected cells can be colony purified and analyzed. Analysis of the transformed mammalian cells may include sequencing of the plasmids that are contained in them. The sequencing may be targeted to the segment encoding the guide RNA and the donor DNA. If a barcode is present, the sequencing may be targeted to the barcode as a surrogate for the guide RNA and the donor DNA. Any method for determining the sequence may be used. For library analysis, a massively parallel sequencing technique can be used. Typically such techniques involve amplification before sequencing, often onto a solid support, such as a bead, slide, or array. Such sequencing techniques typically involve short overlapping reads, and high coverage.

### Methods

According to some embodiments, the present method comprises a method for modifying at least one target site in at least one mammalian cell comprising contacting a mammalian cell with at least one type of vector comprising
(i) at least one donor DNA;
(ii) at least one sequence encoding a first guide RNA;
(iii) at least one sequence encoding a second guide RNA;
(iv) at least one sequence encoding a sequence-specific nuclease.

According to some embodiments, the present system further comprises at least one additional sequence. Additional sequences may encode for additional sequence-specific nucleases, selectable markers, and/or regulatory elements that are useful for gene editing and/or expression. According to some embodiments, the at least one type of vector further comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more additional sequences.

In an exemplary method for modifying at least one target site in at least one mammalian cell, once incorporated into the cell, the at least one sequence-specific nuclease complexes with at least one first guide RNA and cleaves the target gene at at least one target site, at least one sequence-specific nuclease separately complexes with at least one second guide RNA and cleaves the at least one donor DNA thereby generating at least one insertable gene, which is then inserted into the host cell genomic cleavage site. In some embodiments, the at least one insertable gene is inserted at the N-terminal end of a promoter region, an enhancer region, a repressor region, an insulator region, a silencer region, a region involved in DNA looping with the promoter region, a gene splicing region, or a transcribed region to generate a N-terminal tagged fusion protein. In some embodiments, the at least one insertable gene is inserted at the C-terminal end of a promoter region, an enhancer region, a repressor region, an insulator region, a silencer region, a region involved in DNA looping with the promoter region, a gene splicing region, or a transcribed region to generate a C-terminal tagged fusion protein. In some embodiments, the at least one insertable gene is inserted at the C-terminal transcribed region. In some embodiments, the at least one insertable gene is inserted at the N-terminal transcribed region.

In another exemplary method for modifying at least one target site in at least one mammalian cell, once incorporated into the cell, the at least one sequence-specific nuclease complexes with at least one first guide RNA and cleaves the target gene at at least one target site, at least one additional sequence-specific nuclease separately complexes with at least one second guide RNA and cleaves the at least one donor DNA thereby generating at least one insertable gene. In some embodiments, the sequence-specific nuclease is different from the additional sequence-specific nuclease.

According to some embodiments, the at least one type of vector comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more donor DNA. According to some embodiments, upon incorporating into the at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more target sites are modified.

According to some embodiments, the at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more donor DNA comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more sequences encoding at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more gene insertions. According to some embodiments, the at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more gene insertions encodes at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more tags selected from the group consisting of an antibody tag, antibody-epitope tag, fluorescent protein tag, or an affinity purification tag, or combinations thereof. According to some embodiments, the tag is a fluorescent protein.

According to some embodiments, the at least one first guide RNA hybridizes to the at least one target site of the host cell. According to some embodiments, a single first guide RNA hybridizes with a single target site on the host genome. According to some embodiments, a plurality of first guide RNA hybridizes with a plurality of target sites on the host genome. According to some embodiments, the at least one type of vector comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more sequences encoding at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more first guide RNAs. According to some embodiments, the first guide RNA is gene specific. According to some embodiments, the at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more first guide RNAs hybridizes to at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more target sites.

According to some embodiments, if more than one first guide RNA is used then the first guide RNAs may be constructed to be orthogonal.

According to some embodiments, the at least one second guide RNA hybridizes to the at least one target site on the donor DNA. According to some embodiments, a single second guide RNA hybridizes with a single target site on the donor DNA. According to some embodiments, a plurality of second guide RNAs hybridizes with a plurality of target sites on the donor DNA. According to some embodiments, the at least one type of vector comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more sequences encoding at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more second guide RNAs. According to some embodiments, the at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more second guide RNAs hybridizes to at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more target sites.

According to some embodiments, if more than one donor DNA is used then a heterologous cell population may be obtained. According to some embodiments, if more than one donor DNA is used then an isogeneic cell population may be obtained.

According to some embodiments, if more than one second guide RNA is used then the second guide RNAs may be constructed to be orthogonal.

According to some embodiments, the at least one type of vector comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more sequences encoding at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more sequence-specific nucleases.

According to some embodiments, the sequence-specific nuclease is a Cas nuclease. According to some embodiments, the Cas nuclease may be an Cas9 ortholog selected from the group consisting of Streptococcus pyogenes, Staphylococcus aureus, Steptococcus thermophilus, Lactobacillus gasseri, Francisella novicida, Wolinella succinogenes, Sutterella wadsworthensis, gamma proteobacterium, Neisseria meningitidis, Camplyobacteri jejuni, Fibrobacter succinogenes, Rhodobacter speaeroides, Thermus thermophilus, Pyrococcus pyogenes, and Rhodospirillum rubrum, or recombinant hybrids thereof. According to some embodiments, if more than one Cas nuclease is used, each Cas nuclease is a distinct ortholog from each other. Exemplary Cas9 orthologs include Streptococcus pyogenes, Staphylococcus aureus, and Steptococcus thermophiles.

According to some embodiments, if more than one sequence-specific nuclease is used then the sequence-specific nucleases may be constructed to be orthogonal.

According to some embodiments, the at least one type of vector comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more sequences encoding at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more selectable markers. According to some embodiments, the at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more sequences encoding at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more selectable markers encode for at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more drug resistant markers. Examples of drug resistant markers include the puromycin resistant gene, blasticidin resistant gene, or nourothricin resistant gene.

According to some embodiments, the at least one type of vector is a lentiviral vector or a plasmid vector. According to some embodiments, method comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more vectors.

According to some embodiments, the any one of the above described components may be packaged or present in the same vector, different vectors, or any combinations thereof. For example, at least one donor DNA, at least one sequence encoding a first guide RNA, at least one sequence encoding a second guide RNA, at least one sequence encoding a sequence-specific nuclease, at least one sequence encoding a selectable marker, and at least one sequence encoding a regulatory element may be present in the same vector. In another example, at least one sequence encoding a first guide RNA and at least one sequence encoding a sequence-specific nuclease may be present in the same vector. In a further example, at least one sequence encoding a first guide RNA and at least one sequence encoding a selectable marker may be present in the same vector. In a still further example, at least one sequence encoding a second guide RNA and at least one sequence encoding a selectable marker may be present in the same vector. In an additional example, at least one donor DNA and at least one sequence encoding a regulatory element may be present in the same vector. In an additional example, at least one donor DNA and at least one sequence encoding a selectable marker may be present in the same marker. It is understood that if multiples of any component, namely, donor DNA, first guide RNA, second guide RNA, sequence-specific nuclease, selectable marker, and/or regulatory element are used herein, each one of the multiple may be present in different vectors.

According to some embodiments, upon contacting the at least one donor DNA incorporates into the at least one target site. According to some embodiments, upon incorporating into at least one target site of at least one cell expresses at least one donor DNA. According to some embodiments, at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more target sites are modified on at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or cells.

According to some embodiments, the present method further comprises the step of selecting for the selectable marker and expanding the cells. According to some embodiments, selecting for the selectable marker comprises treating the cells with a corresponding selective agent or screening for the selectable marker. According to some embodiments, the selective agent is a drug. According to some embodiments, the drug is puromycin, blasticidin, or nourothricin.

According to some embodiments, the sequence(s) encoding first guide RNA(s) contacts the cell prior to the donor DNA(s), the sequence(s) encoding second guide RNA(s), and the sequence(s) encoding sequence-specific nuclease(s). According to some embodiments, the donor DNA(s), the sequence(s) encoding second guide RNA(s), and the sequence(s) encoding sequence-specific nuclease(s) contacts the cell after the sequence(s) encoding first guide RNA(s).

According to some embodiments, after the sequence(s) encoding first guide RNA(s) contacts the cell, the present method further comprises the step of selecting for the selectable marker and expanding the cells.

According to some embodiments, after the donor DNA(s), the sequence(s) encoding second guide RNA(s), and the sequence(s) encoding sequence-specific nuclease(s) contacts the cell, the present method further comprises the step of selecting for the selectable marker and expanding the cells. According to some embodiments, the method further comprises contacting the cell again with the donor DNA(s), the sequence(s) encoding second guide RNA(s), and the sequence(s) encoding sequence-specific nuclease(s). According to some embodiments, after the donor DNA(s), the sequence(s) encoding second guide RNA(s), and the sequence(s) encoding sequence-specific nuclease(s) contacts the cell, the present method again comprises the step of selecting for the selectable marker and expanding the cells. Selection and expansion may be repeated as necessary until the desired cell population is obtained.

According to some embodiments the present invention is effective to obtain an isogeneic cell population. According to some embodiments the present invention is effective to obtain an heterogeneic cell population. According to some embodiments the present invention is effective to obtain at least one isogeneic cell population from a heterogeneic cell population.

According to some embodiments, the present invention is effective to increase or enhance targeting, cleavage efficiency, integration efficiency, fusion efficiency, or any combination thereof, at least about 1%, at least about 5%, at least about 10%, at least about 20%, or at least about 30%, or at least about 40%, or at least about 50%, or at least about 60%, or at least about 70%, or at least about 80%, or at least about 90% or up to and including a 100% increase or any increase between 1-100%.

The disclosed methods can be used for genome-wide protein labelling, expression marking, disruption of protein expression, protein re-localization, alteration of protein expression, or high throughput screening. In accordance with these embodiments, the method would allow for both speed and precision in applications including but not limited to antibody staining of fixed cells or tissues, live imaging of protein in cells or tissues, protein capture or affinity purification for protein complex identification, cell-type lineage tracing or labeling, and production of transgenic organisms with multiple different fusions to an individual gene.

According to some embodiments, the present methods are scalable. According to some embodiments, the present scalable methods are useful for high throughput gene modification. According to some embodiments, the present scalable methods are useful for high throughput genome-wide protein labelling, expression marking, disruption of protein expression, protein re-localization, alteration of protein expression, or screening. The methods that described herein can be used to rapidly generate protein variants for functional characterization of any protein-of-interest. The methods can be used for domain deletion, amino acid replacement, and protein tagging, all of which are common approaches for elucidating protein function.

### EXAMPLES

### Example 1

### Scalable Single-Locus Insertion Systems

Example 1.A) Materials

Host Cell. HEK293T human cell lines were used as mammalian host cells.

Cas9. RNA-guided endonuclease Streptococcus pyogenes Cas9 (spCas9) was obtained.

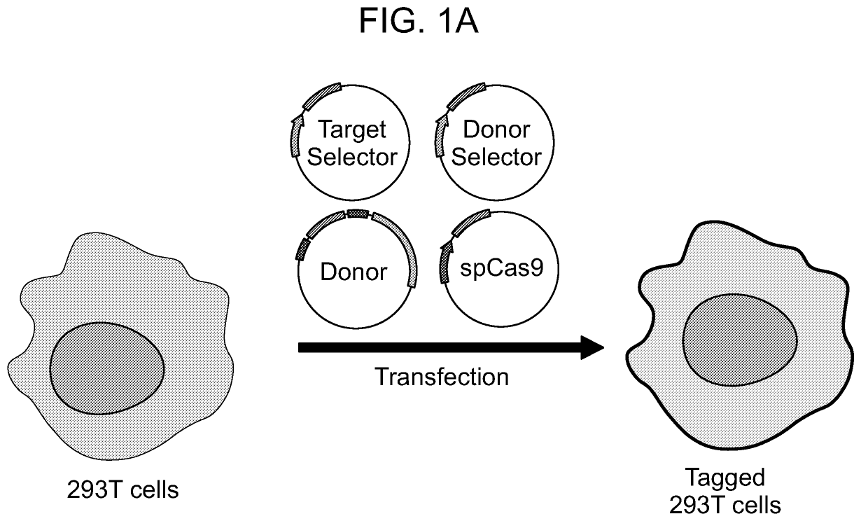

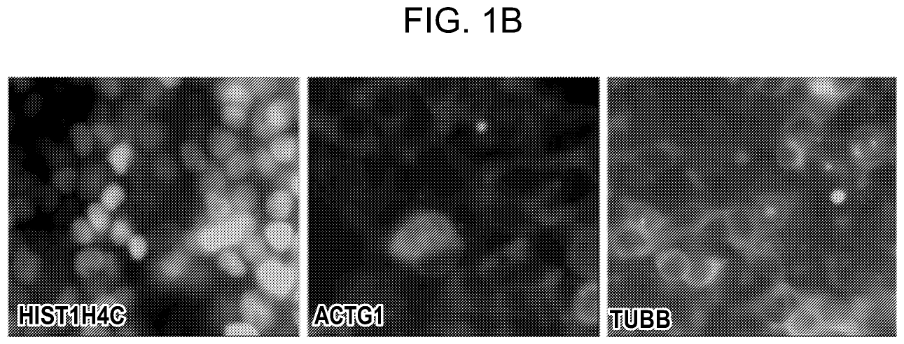

Constructs. To successfully tag and express targeted proteins in mammalian cells, appropriate constructs were designed or obtained, namely, a plasmid encoding a target selector gRNA, a plasmid encoding donor selector gRNA, and donor plasmid(s) encoding the DNA sequence of interest to be hybridized to the target locus. A representative schematic can be seen in FIG. 1A.

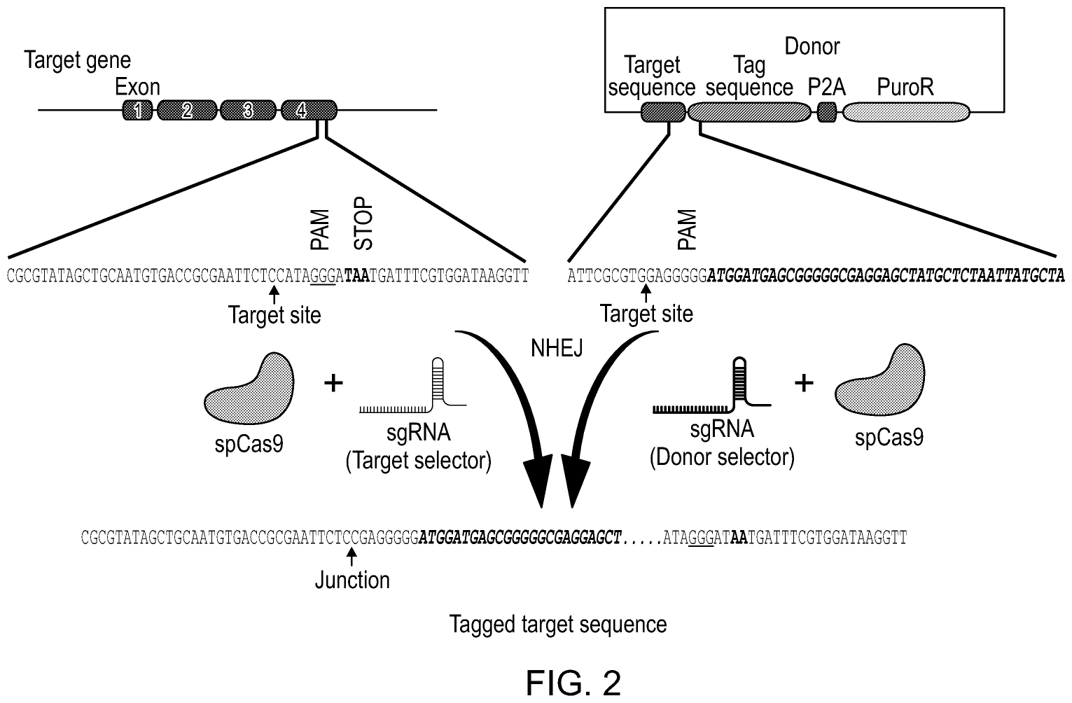

Donor plasmid selection. Donor plasmids were designed or obtained that individually encode a DNA sequence of interest. Three sequences were of interest, the DNA encoding HIST1H4, ACTG1, and the TUBB gene. Donor plasmids also encoded with an appropriate fluorescent tag sequence, 2A “self cleaving” sequence (p2A), and puromycin resistant gene. A representative schematic can be seen in FIG. 2.

The donor plasmid was carefully selected in consideration of the Cas9 molecular mechanism and the reading frame of the target gene. Cas9 can cut DNA three base pairs upstream of the PAM sequence, meaning that there are three potential reading frames (Frame 0, +1, +2). Therefore, the donor plasmid was selected so that when cut and fused with the endogenous locus, the inserted donor product will be in frame with the upstream target gene. A representative schematic can be seen in FIG. 3.

Example 1.B) Methodology and Molecular Mechanism

Briefly, host cells were transfected with the plasmids and spCas9 to obtain a pool of tagged 293T cells. A representative schematic can be seen in FIGS. 1A and 1B.

Once all constructs are within the cell spCas9 complexes with the target selector gRNA and makes a double strand break within the host gene that the desired DNA sequence is targeted to hybridize to. Contemporaneously, spCas9 complexes with the donor selector gRNA and linearizes the donor plasmid. During the repair of the DNA break at the target gene, the linearized donor can become inserted into the locus via Non-Homologous End-Joining (NHEJ), which creates a fusion between the cut gene and the donor molecule. A representative schematic can be seen in FIG. 2.

Cells were enriched through treatment of puromycin, obtaining cells with properly knocked in donor molecule, expressing the tagged protein of interest. FIG. 1B shows YFP signals from cells tagged at the YFP HIST1H4, ACTG1, or the TUBB gene.

### Example 2

### Scalable Single-Locus, Multi-Insertion Systems

Example 2A) Target Selector Library and target Selector Cell Library formation

A population target selector plasmids were generated, each plasmid encoding sequences for one of four gene-specific gRNA, individually, HISTIH4C, CCT7, ACTC1, or NEUROD1, together with a C-terminus YFP tag, 2A “self cleaving” sequence (p2A), and a blasticidin resistant gene, to obtain the target selector library.

Lentiviral packaging was separately prepared or obtained. Lentivirus preparation is known in the art and as briefly described herein.

The target selector library was then converted into lentivirus, and used to infect host cells at a low multiplicity of infection (MOI) so most cells received a single lentivirus. Cells were then treated with blasticidin until cells that expressed the desired gRNA were selectable. Once selected and expanded under appropriate conditions, the target selector cell library was obtained.

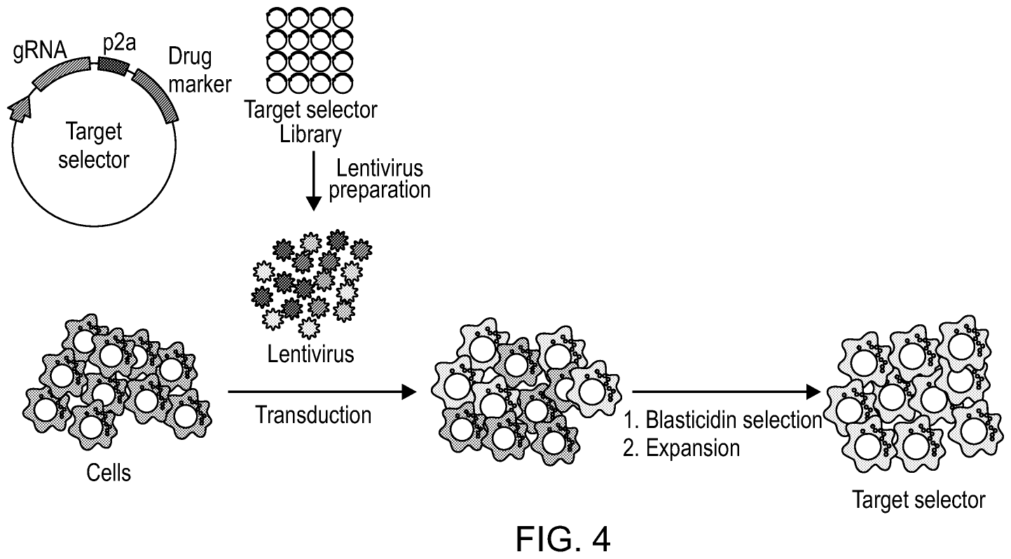

A representative schematic is as shown in FIG. 4

Example 2B) Multi-Tagged Heterogenetic Cell Population

The remaining components, spCas9, donor plasmid and donor selector gRNA were constructed or obtained as described in Example 1, and were added to the mixed pool of the target selector cell library and subjected to transfection through the use of lipofectamine 2000. Once transfected, and the cells with proper targeted knock-in expressed the drug resistance marker, cells were enriched by treating with puromycin and expanding to obtain a multi-tagged heterogenetic cell population wherein YFP was knocked-into various genes, with each cell knocking-in YFP into a single gene.

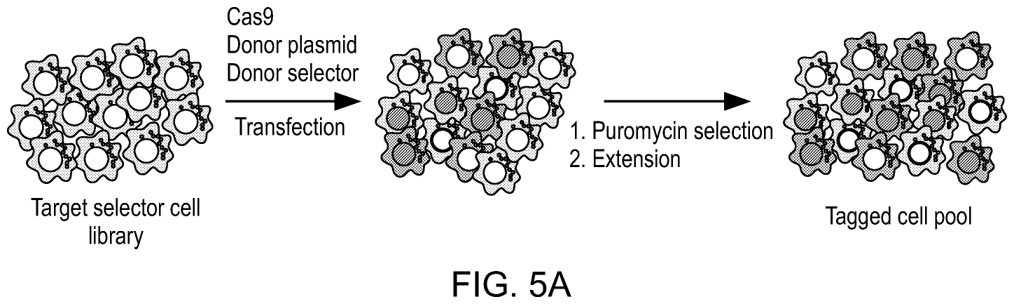

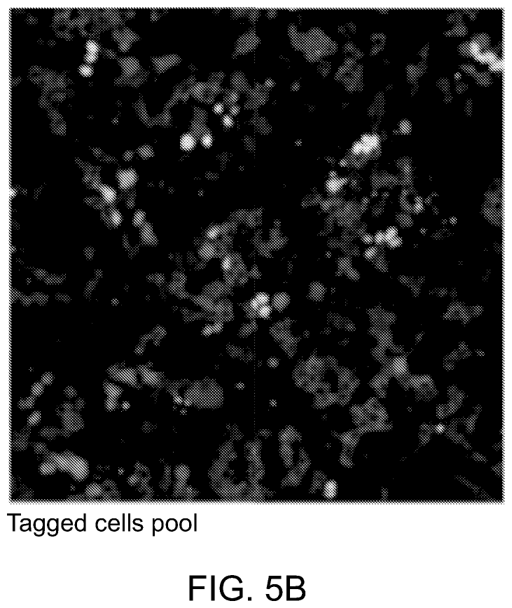

A representative schematic is as shown as in FIG. 5A. FIG. 5B. shows the mutant cell pool expressing mixed fluorescent signals.

Example 2C) Multi-Tagged Isogeneic Cell Population Isolation and Sequencing

The mixed knock-in pool, i.e., the multi-tagged heterogenetic cell population, was sorted and plated at one cell per well in a 96-well (microwell) plate using fluorescence-activated cell sorting (FACS). Cells were cultured in appropriate medium until the wells were 100% confluent by a homogenous clonal population. Homogenous isogeneic population of cells were obtained, with each well having a different gene tagged with YFP.

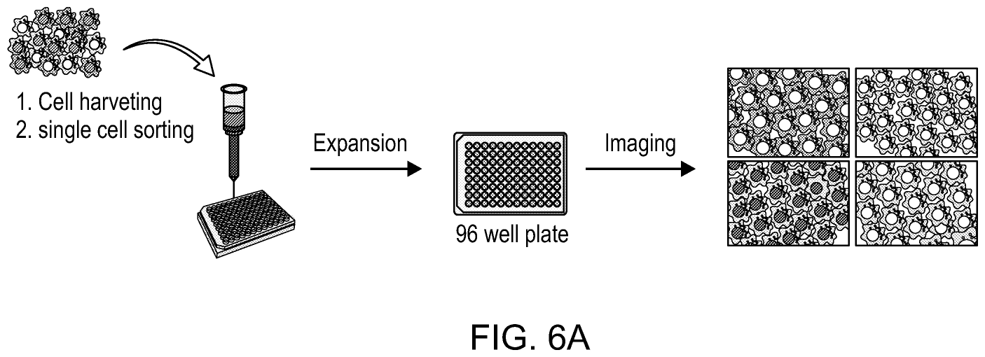

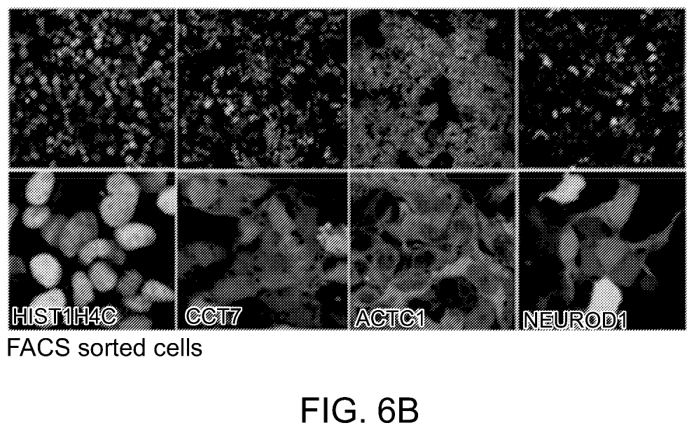

A representative schematic is as shown in FIG. 6A, FIG. 6B shows FACS sorted cells expressing YFP tagged protein products of HISTIH4C, CCT7, ACTC1, or NEUROD1.

gRNA sequencing and PCR verification for the target gene and donor sequence junction may be individually performed to verify the identity of the clonal population inside each well. For high throughput approaches, a Cartesian Pooling-Coordinate Sequencing approach may be used.

### Example 3

### Variations for Scalable Gene Insertion Systems

The methods and systems described herein may be used to insert any DNA sequence into any defined genomic locus. In addition to tagging fluorescent proteins, the described systems and methods may knock-in epitope tags, small molecule regulated degrons, or effector proteins in-frame with the target gene of interest. Donor vectors may be constructed for example with DNA encoding for FLAG, BirA, MBP, and polyH to obtain flag-tagged cells, Bir-A tagged cells, MBP tagged cells, and His tagged cells, respectively, when the methods described herein are performed.

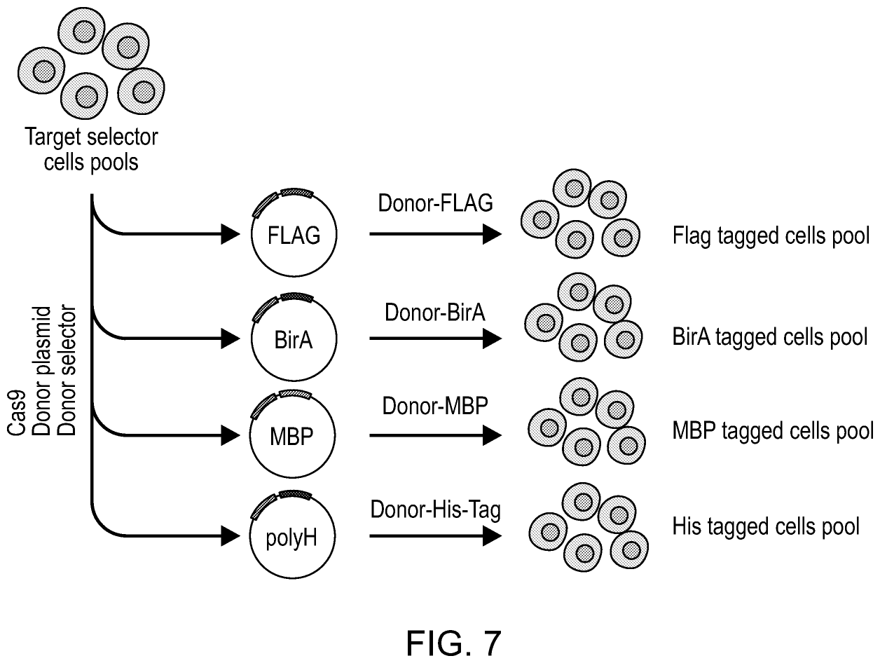

A representative schematic is as shown in FIG. 7.

### Example 4

### Applications for Scalable Gene Insertion Systems

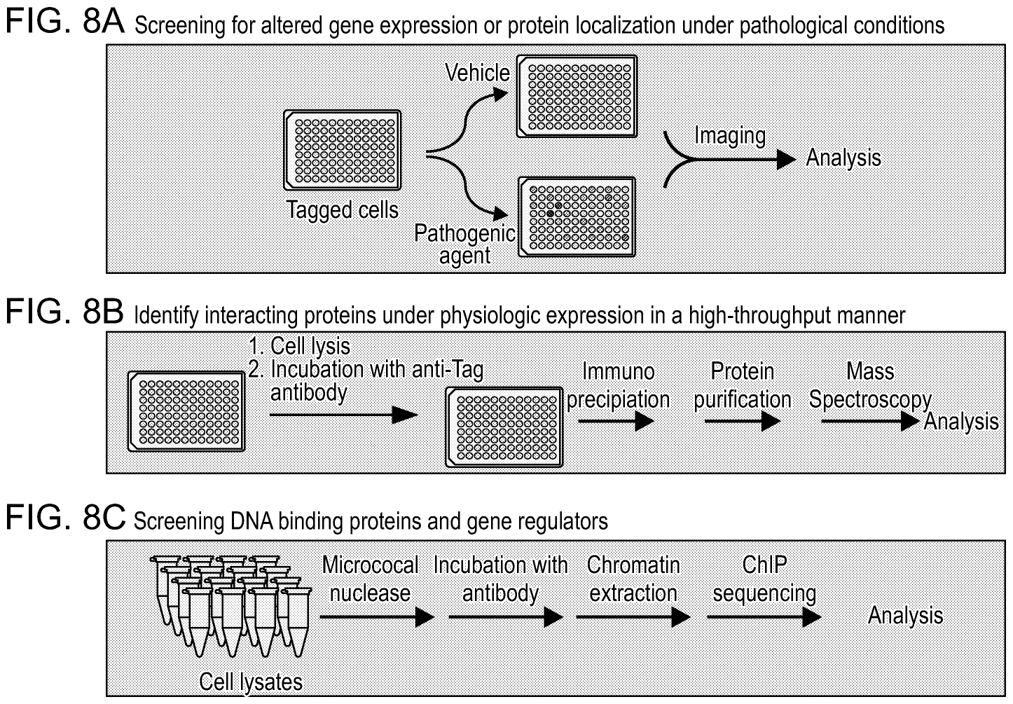

Clonal lines expressing the desired gene insertion(s) may be obtained for any downstream assay of interest obtained. A representative example can be seen in FIGS. 8A, 8B, and 8C.

Example 4A) Clonal Lines May Be Screened for Altered Gene Expression or Protein Localization Under Pathological Conditions.

The methods and systems described herein may be performed to generate multi-YFP tagged cells. Cells may be cloned into a control and test group wherein the control group is treated with a vehicle control and the test group is treated with a pathogenic agent. Imaging of the clonal populations study groups may provide information of the pathogeneic agent's effect on the tagged proteins' localization or expression level.
Example 4B) Clonal Lines May Be Analyzed to Identify Interacting Proteins Under Physiologic Expression in a High Throughput Manner.

The methods and systems described herein may be performed to generate epitope tagged cells. Once the epitope-tagged cells are obtained, the cells may be lysed and incubated with anti-Tag antibody, immunoprecipitated, purified and undergo mass spectroscopy to isolate protein complex(es) and/or discover any novel interacting partners.

Example 4C) Clonal Lines May Be Screened to Identify DNA Binding Proteins and Gene Regulators.

The methods and systems described herein may be performed to generate a tagged cells, which may be lysated and digested with micrococal nuclease, incubated with an antibody, undergo chromatin extraction via immunoprecipitation and undergo ChIP sequencing to identify nucleic acids with which the tagged proteins interact in high throughput.

### Example 5

### Double Cas Scalable Gene Insertion Systems

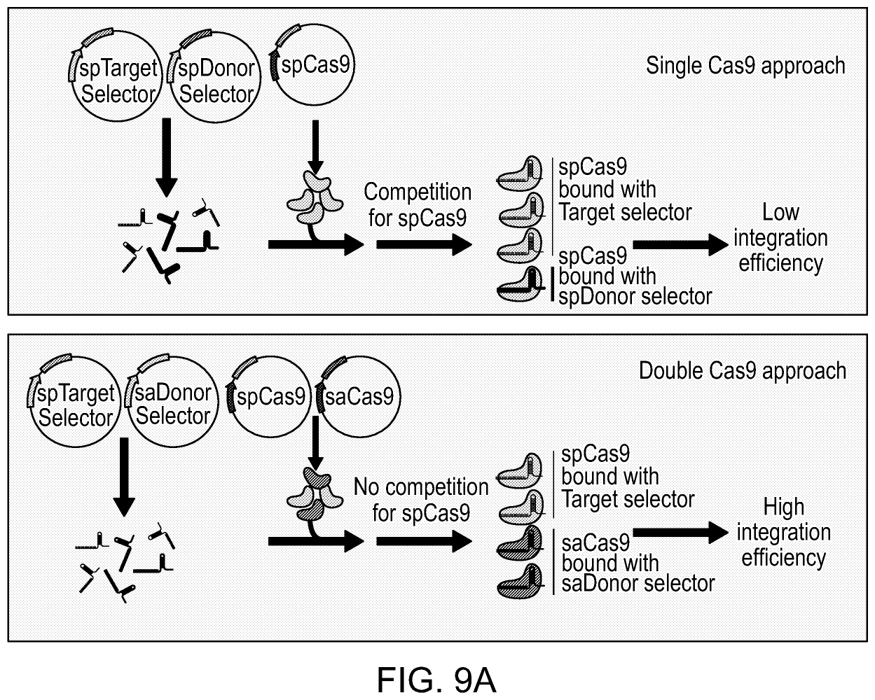

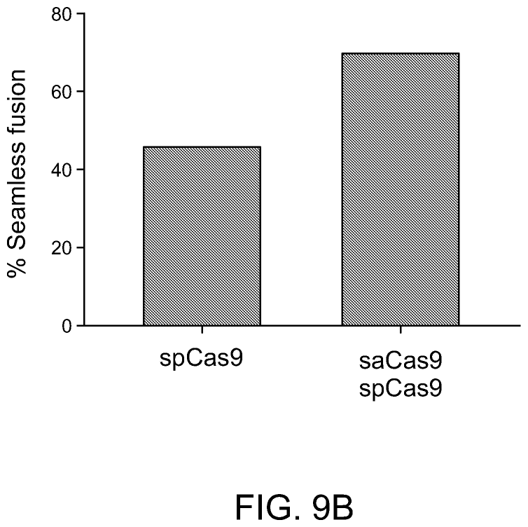

In the methods and systems described herein, it is hypothesized that spCas9 is competed for by target selector gRNA and donor selector gRNA within the cell. Two orthogonal protein pairs were constructed to avoid competition: spCas9/spTarget Selector; and Staphyolococcus aureus Cas9 (saCas9)/saDonor Selector. A representative schematic is as shown in FIG. 9A.

When compared to a single Cas system, for example as described in Example 1 above, the results, as shown in FIG. 9B, indicate that the double Cas system lead to an increase in fusion percent. It can be concluded that competition was prevented or lessened between the gRNAs and more efficient cutting of the donor and the target locus of interest occurred.

### Example 6

### Scalable Multi-Locus Multi-Insertion Systems

Example 6A) Multiplex Target Selector and Multiplex Target Selector Cell Library

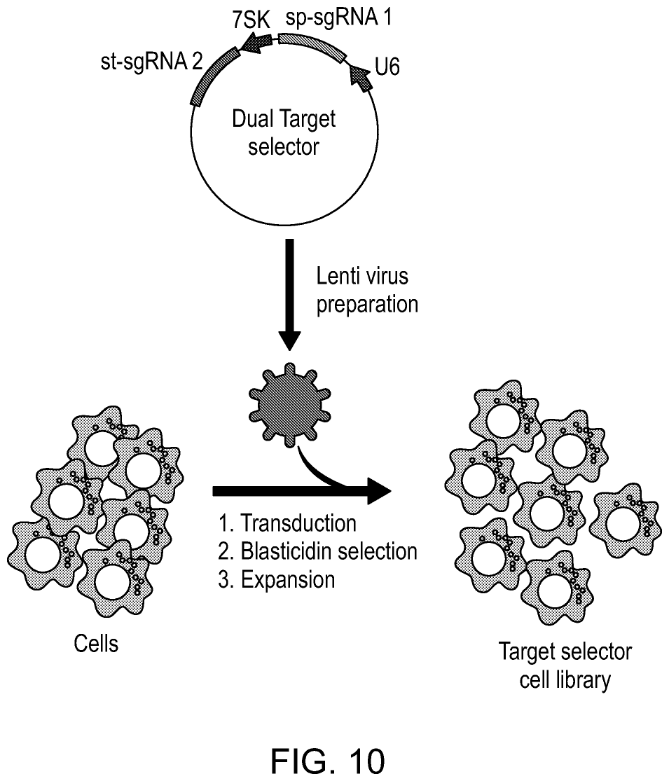

To target more than a single locus, a dual target selector may be constructed to express two orthogonal gRNAs against two target sites of interest. The dual target selector may encode for sp-sgRNA 1, st-sgRNA 2, and corresponding promotors, U6 and 7SK, respectively. The dual target selector may be packaged into a lentivirus preparation. The host cells may be infected with the lentivirus at a low MOI, and the stably transduced cells may be selected using blasticidin. Upon expansion of the selected cells, a dual target selector cell library may be obtained. A representative schematic is as shown in FIG.10.

Example 6B) Multi-Gene Tagged Isogeneic Cell Population

Briefly, the multiple target selector library cells may next be treated with rounds of transfection and drug selection to obtain a multi-gene tagged isogeneic cell population.

Round I. The dual target selector library cells may be transfected with spCas9 (which complexes with sp-gRNA 1, encoded by the target selector cell library), saDonor selector encoding sa-gRNA, saCas9 protein (which complexes with sa-gRNA), and a donor plasmid encoding for a GFP2 tag and nourothricin resistant gene. The transfected cells are treated with nurothricin and expanded to obtain a single gene tagged cell pool.

Round II. The single gene tagged cell pool may next be transfected with stCas9 (which complexes with st-gRNA 1 encoded by the target selector cell library), saDonor selector encoding sa-gRNA, saCas9 protein (which complexes with sa-gRNA), and a donor plasmid encoding for a RFP tag and puromycin resistant gene. The double transfected cells are treated with puromycin and expanded to obtain a dual gene tagged cell pool.

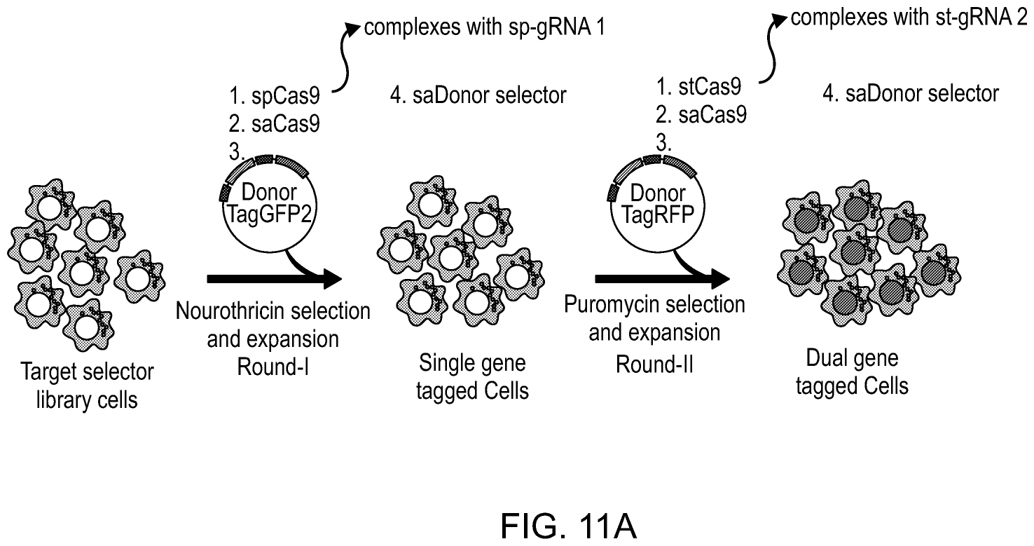

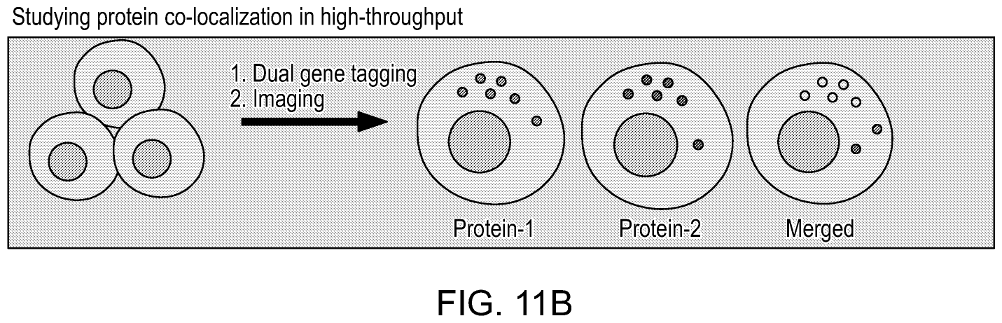

A representative schematic is as shown as in FIG. 11A.

### Example 7

### Applications of Scalable Multi-Locus Multi-Insertion Systems

It is hypothesized that cells with multiple genomic loci tags can be used to study dynamics between several proteins within live cells. For example, once dual gene tagged cells are obtained, imaging may be performed to study expression of a first protein in the population of cells, expression of a second protein in a populations of cells, and co-localization of expression of both proteins in a population of cells.

A representative schematic is as shown as in FIG. 11B.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

What is claimed is:

1. A system for modifying a plurality of target sites in a mammalian cell, wherein the system comprises at least one type of vector comprising:
(i) a plurality of donor DNAs;
(ii) a plurality of first sequences encoding a plurality of first guide RNAs that hybridize to a plurality of target sites in the cell;
(iii) a plurality of second sequences encoding a plurality of second guide RNAs that hybridize to the plurality of donor DNAs; and,
(iv) a third sequence encoding a sequence-specific nuclease.

2. The system of claim 1, wherein two, three, or all four of (i)-(iv) are present in the same vector.

3. The system of claim 1, wherein each of (i)-(iv) are present in different vectors.

4. The system of claim 1, wherein the plurality of first sequences encoding a plurality of first guide RNAs hybridize to a plurality of different target sites in the cell.

5. The system of claim 1, wherein the at least one type of vector comprises lentiviral vectors or plasmid vectors.

6. The system of claim 1, wherein the at least one type of vector comprises at least one additional sequence encoding at least one additional sequence-specific nuclease.

7. The system of claim 6, wherein the sequence-specific nuclease and the at least one additional sequence-specific nuclease are different.

8. The system of claim 6 or 7, wherein the at least one type of vector encodes two sequence-specific nucleases binding to the first guide RNAs or the second guide RNAs, respectively.

9. The system of any one of claims 1-8, wherein at least one or all of the sequence-specific nucleases are a Cas nuclease.

10. The system of any one of claims 1-9, wherein each of the Cas nuclease is a Cas9 ortholog individually selected from the group consisting of Streptococcus pyogenes, Staphylococcus aureus, Steptococcus thermophilus, Lactobacillus gasseri, Francisella novicida, Wolinella succinogenes, Sutterella wadsworthensis, gamma proteobacterium, Neisseria meningitidis, Camplyobacteri jejuni, Fibrobacter succinogenes, Rhodobacter speaeroides, Thermus thermophilus, Pyrococcus pyogenes, and Rhodospirillum rubrum, or recombinant hybrids thereof, preferably wherein each Cas nuclease is a distinct ortholog from each other.

11. The system of claim 10 wherein at least one of the Cas nuclease is Streptococcus pyogenes Cas9 (SpCas9).

12. The system of claim 10, wherein the Cas nuclease and the second Cas nuclease are Streptococcus pyogenes Cas9 (SpCas9) and Staphylococcus aureus Cas9 (SaCas9), respectively.

13. The system of claim 10, wherein the Cas nuclease, the second Cas nuclease, and at least one additional Cas nuclease are Streptococcus pyogenes Cas9 (SpCas9), Staphylococcus aureus Cas9 (SaCas9), and Steptococcus thermophiles (StCas9), respectively.

14. The system of claim 1, wherein the at least one type of vector further comprises a plurality of additional sequences encoding a plurality of selectable markers, optionally, comprising multiple distinct markers.

15. The system of claim 14, wherein the plurality of selectable markers encodes a plurality of drug resistant markers, optionally multiple distinct drug resistant markers.

16. The system of claim 15, wherein the plurality of sequences encodes a plurality of drug resistant markers individually selected from the group consisting of puromycin resistant genes, blasticidin resistant genes, and or nourothricin resistant genes.

17. The system of claim 14, wherein the system further comprises a means for selecting for the selectable marker and a means for expanding after selection.

18. The system of claim 1, wherein the plurality of donor DNAs comprises a plurality of sequences encoding for a plurality of gene insertions, optionally comprising multiple distinct insertions.

19. The system of claim 18 wherein the plurality of sequences encoding for a plurality of gene insertions encode proteins individually selected from the group consisting of an antibody tag, antibody-epitope tag, fluorescent protein tag, an affinity purification tag, a protein-protein interaction domain, a chemically induced protein-protein interaction domain, an enzyme, a recombinase site, a protein stability regulating tag, a spatial localization sequence, DNA/RNA targeting protein, or a combination thereof.

20. The system of claim 1, wherein the system is for modifying a plurality of target sites in a population of mammalian cells, optionally, different mammalian cells.
